(19) United States
(12) Patent Application Publication
(10) Pub. No.: US 2009/0030293 A1
(43) Pub. Date: Jan. 29, 2009

(54) SENSING DEVICE, APPARATUS AND SYSTEM, AND METHOD FOR OPERATING THE SAME

(75) Inventors: Jonathan Mark Cooper, Glasgow (GB); David Robert Sime Cumming, Glasgow (GB); Nicholas Wood, Bedford (GB); Lei Wang, London (GB)

Correspondence Address: TOLER LAW GROUP, 8500 BLUFFSTONE COVE, SUITE A201, AUSTIN, TX, 78759, US

(73) Assignee: The University Court of the University of Glasgow, Glasgow (GB)

(21) Appl. No.: 11/815,841

(22) PCT Filed: Feb. 10, 2006
(86) PCT No.: PCT/GB2006/000465

§ 371 Date: Aug. 8, 2007

(30) Foreign Application Priority Data
Feb. 11, 2005 (GB) 0502886.5
Mar. 17, 2005 (GB) 0505512.4
Mar. 17, 2005 (GB) 0505513.2

(51) Int. Cl. A61B 5/07; A61B 5/055; H04L 5/16; A61B 5/1455

(52) U.S. Cl. 600/302; 600/348; 600/310; 375/219

(57) ABSTRACT

A sensing device, apparatus and system, and method for operating the same are provided. The sensing apparatus includes a first module and a second module. The first module includes a controller, a transmitter and an array of sensor elements. The controller is capable of activating one or more sensor elements in the array independently of others in the array, in order to obtain a sensor output from the array at different times by using different sensor elements in said array. The transmitter is configured to transmit sensor data derived from the sensor output from the first module to a receiver of the second module. Each sensor element is a biological sensor for detecting the presence of the same analyte in the environment in which the sensor array is to be deployed.

### CLAIM OF PRIORITY

This application claims priority to International Application Number PCT/GB2006/000465 filed Feb. 10, 2006, which claims priority to Great Britain Patent Application No. 0502886.5, filed Feb. 11, 2005; Great Britain Patent Application No. 0505513.2, filed Mar. 17, 2005; and Great Britain Patent Application No. 0505512.4, filed Mar. 17, 2005, all of which are expressly incorporated herein by reference in their entirety.

### FIELD OF THE DISCLOSURE

The present disclosure is generally related to a sensing device, a sensing apparatus and a sensing system. The disclosure also relates to methods for operating such a device, apparatus and/or system. The disclosure is particularly, but not exclusively, concerned with gathering biomedical data and/or information.

### BACKGROUND

Known swallowable capsules incorporate a miniature camera as a sensor, the camera obtaining a series of images of the gastrointestinal (GI) tract during its transit through the GI tract. The images obtained by the camera are transmitted over a radio link to a base station. The series of images is then reviewed by a skilled operator, who looks for abnormalities in the GI tract. Such imaging can provide useful diagnostic information, but requires a great deal of time of the skilled operator for each patient.

### BRIEF DESCRIPTION OF THE DRAWINGS

Further features and aspects of the disclosure can be found in the accompanying description and the appended claims. Embodiments disclosed herein will now be described, by way of example, with reference to the accompanying figures, in which:

FIG. 1 is a schematic diagram of a sensing device;

FIG. 2 is a schematic diagram of a system including a sensing device and a base station;

FIG. 3 is a schematic diagram of another embodiment of a system including a sensing device with a receiver and a base station;

FIG. 4 is a graph showing variation in pH as the sensing device travels through the digestive system;

FIG. 5 is a schematic diagram of a sensor and surrounding circuitry for adjusting its dynamic range;

FIG. 6 shows a routine for adjusting the dynamic range of a sensor;

FIG. 7 shows a routine for assigning actual physical value to the sensor output;

FIG. 8 shows a routine for auto-zeroing the sensor output or referencing it to a desired value;

FIG. 9 shows a routine for compensating for drift in the sensor output over time;

FIG. 10(a) shows measured changes in the source voltage of an ISFET resulting from the measured and modeled threshold voltage drift of the ISFET which is shown in FIG. 10(b);

FIG. 11(a) is a graph showing ISFET threshold voltage response as measured in response to a change in solution pH and FIG. 11(b) shows the same threshold voltage after drift compensation has been applied;

FIG. 12 is a schematic diagram of a sensing device;

FIG. 13 is a schematic diagram of a system including a sensing device and a base station;

FIG. 14 is a schematic diagram of another embodiment of a system including a sensing device with a receiver and a base station;

FIGS. 15(a) to (c) are schematic diagrams showing possible arrangements of modular systems;

FIG. 16 is a schematic diagram showing the components of a sensing device;

FIG. 17 is another schematic diagram showing how the components of the sensing device in FIG. 16 are split onto separate chips or circuit boards;

FIG. 18 is a perspective view of the electronic components of a sensing device;

FIG. 19 is a perspective view showing the sensing device's electronic components and surrounding capsule casing when dissembled;

FIG. 20 is a flow chart showing the processing of received data by the second module;

FIG. 21 is a time line showing zero-periods and data packets and the time taken for data acquisition and other processes to be carried out by the second module;

FIG. 22 is a graph showing data bits and a noise spike against time;

FIG. 23 is a top down view of the a capsule having a helical groove;

FIG. 24 is a top down view of a capsule having a helical projection;

FIG. 25A is a schematic view of the external surface of a sensing device;

FIG. 25B is a schematic view of the external surface of an alternative sensing device;

FIG. 25C is a schematic view of the external surface of an alternative sensing device;

FIG. 26 is a schematic view of a sensor element array of a sensing device;

FIG. 27 is a schematic diagram showing a sensing system of a first module and a second module;

FIG. 28 is a plan view of a sensing element; and

FIG. 29 is a cross sectional view of the sensing element of FIG. 28.

### DETAILED DESCRIPTION OF THE DRAWINGS

The present disclosure is particularly useful in systems where a swallowable capsule with a sensor is swallowed by a patient and transmits gathered data from inside the body to a base station outside the body via a radio or other communication link. However it is not limited to this application and may also be used on a sensing device designed for implantation into the human body. It may also be used in topical application, e.g. in wound dressings. It may also be used with animals, especially but not limited to agricultural livestock, such as cattle sheep and pigs. Application not only to mammals, but also to non-mammals, e.g. fish at fish farms, would also be possible.

The present disclosure includes several related developments, as set out below. For each development, there are several aspects. It is to be understood that it is possible to combine aspects of any development with each other, unless the context demands otherwise. Similarly, it is possible to combine preferred and/or optional features singly or together with any of the aspects of any development, unless the context demands otherwise.

First, it may be advantageous to provide alternative sensors in a swallowable capsule. In particular, some disorders of the CI tract are difficult to detect using a camera sensor. For example, bleeding in the GI tract is a common symptom of several diseases such as Crohn's disease, ulcerative colitis, ulcers and cancer. Bleeding in the GI tract can go unnoticed until it reaches a scale where other symptoms appear, e.g. anaemia, or if fresh blood appears in the stool. By this time, the disease has usually reached an advanced stage. In the case of bowel cancer, polyps often bleed before they become cancerous. Consequently, if they can be detected early, the polyps can be safely removed and the cancer treated successfully.

There are faecal occult blood (FOB) tests, for testing for the presence of blood in stool. These are generally based on the peroxidase-like behaviour of haemoglobin or are based on immunoassays.

One type of FOB test uses a guaiac resin impregnated card. Guaiac resin (extracted from trees) changes colour in the presence of oxidising agents. Such tests utilise the fact that haemoglobin catalyses the oxidation of the phenolic compound in guaiac resin (alpha guaiaconic acid) by hydrogen peroxide to form a highly conjugated blue quinone compound. In guaiac-based FOB tests samples of stool are spread by the patient on a card impregnated with guaiac resin. Two samples from each of three stools are typically required to be collected before the card is sent for analysis. In the analysis laboratory, a hydrogen peroxide developer solution is applied to the card and, if blood is present in the sample, a blue-green colour is the result.

The FOB test described above is of use in screening tests, where patients receive the test through the mail, or from their local doctor, take and apply their own samples to the card, and return the card to the laboratory for analysis. The take-up of such tests is variable, particularly amongst the elderly, and amongst people from certain ethnic or social backgrounds, probably due to the unpleasant nature of taking the samples and applying them to the cards.

In a particular embodiment, a sensing apparatus including a first module and a second module is disclosed. The first module has a controller, a transmitter and an array of sensor elements. The controller is capable of activating one or more sensor elements in said array independently of others in the array, in order to obtain a sensor output from said array at different times by using different sensor elements in the array. The transmitter is configured to transmit sensor data, derived from the sensor output, from the first module to a receiver of the second module, wherein each sensor element is a biological sensor for detecting the presence of the same analyte in the environment in which the sensor array is to be deployed. In an illustrative embodiment, the first module is adapted:

(i) to be swallowable, for passage through the human or animal body;

(ii) to be implantable in the human or animal body; or

(iii) to be placed at a surface location of the human or animal body.

For application (i), certain limits may be placed on the physical dimensions and shape of the first module. With respect to the shape, typically the first module is elongate with an aspect ratio of 2.5:1 or more, preferably 3:1 or 4:1 or more. Of course, the particular size is dependent on the GI tract through which the first module should pass. For application (ii), fewer general limits may be placed on the size or shape of the first module. However, for both (i) and (ii), the first module should be formed of biocompatible and/or non-toxic materials. For application (iii), it is preferred that the first module has a flat form, optionally a flexible form. For example, the first module may be provided at a wound site on the body, preferably on or within a wound dressing.

In a particular embodiment, each sensor element is activatable only once to attempt to detect the presence of the analyte in the environment. In this way, each sensor element may only be capable of operation once. For example, the sensor elements may rely on a chemical reaction using at least one reagent, the use of the reagent in a sensor element for a measurement meaning that the sensor element cannot carry out a further measurement.

The sensor output may correspond to an analyte condition of at least one of: analyte present; analyte not present; a quantitative measure of the concentration of analyte detected. Thus, each sensor element may be capable of providing a measure of concentration of the analyte. However, in certain embodiments, it may be sufficient that each sensor element is capable only of determining whether the analyte concentration is above a certain threshold (e.g., analyte present) or below a certain threshold (e.g., analyte not present).

In a particular embodiment, the analyte includes blood, haemoglobin, another component of blood, or a degradation product of blood. Alternatively, the analyte may include other body fluids or components thereof, such as lumen, digestive enzymes, food or the products of food digestion, or wound fluid.

In a particular embodiment, activation of a sensor element in the array allows analyte present in the environment of the sensor element to catalyse a chemical reaction between a first reagent and a second reagent, detection of the chemical reaction by the sensor element determining the sensor element output. For example, each sensor element includes a reagent space containing at least said first reagent. This reagent space may also contain said second reagent. The second reagent may be in contact with said first reagent. The first and second reagents may take the form of layers in contact with each other, of islands of one reagent within another, or of particles of one reagent within another. In general, the form of contact between the two reagents will depend on the reactivity of the two reagents with each other in the absence of analyte, and thus the useful shelf-life of the sensor element.

In a particular embodiment, the reagent space is separated from an electrolyte space by a semipermeable membrane. The semipermeable membrane may be permeable to oxygen, oxygen ions, protons, or other predetermined species. The electrolyte space typically has a working electrode, a counter electrode and optionally a reference electrode, the electrodes being in electrical contact with electrolyte in the electrolyte space. In this way, the electrodes can be used to monitor a reaction between the first and second reagents in the reagent space, by virtue, for example, of oxygen or oxygen ions produced by the reaction between the first and second reagents.

In a particular embodiment, the reagent space is exposable to the environment on activation of the sensor element. Each sensor element may include a cover member for covering said reagent space, the cover member being at least partially removable to allow exposure of said reagent space. Preferably, the cover member is at least partially removable by application of an electrical voltage to said cover member. The electrical voltage may trigger at least one of corrosion, dissolution, melting, sublimation and breakage of said cover member.

In a particular embodiment, the first reagent includes alpha guaiaconic acid, or derivative thereof. In a particular embodiment, the second reagent is a mediator capable of oxidising the first reagent in the presence of a catalyst.

In a particular embodiment, the sensor array is provided at an outer surface of the first module, so as to be provided in contact with the environment in which the first module is to be deployed. In this way, each sensor element may be directly exposed to the environment (at least at the time of activation) without requiring fluid from the environment to travel along channels or conduits in the device. This embodiment may be useful, since some regions of the GI tract (e.g. the colon) have contents that are substantially solid and compacted, and thus difficult to flow.

The sensor array may be formed on a common substrate. For example, each sensor element may be formed using photolithography techniques. The substrate may be planar, for example a silicon single crystal substrate. The substrate may be flexible, in order to be fitted onto a curved outer surface of the first module. The substrate may itself be the outer casing of the first module.

In a particular embodiment, the sensor array may include at least four sensor elements. In other particular embodiments, the array has at least five, at least six, at least seven, at least eight, at least nine, at least ten, at least twelve, at least fourteen, at least sixteen, at least eighteen, at least twenty, at least twenty five, at least thirty, at least thirty five, at least forty, at least forty five or at least fifty sensor elements.

In a particular embodiment, the controller is operable to activate the sensor elements at predetermined time intervals.

The sensor array of the first module may form a first sensor. The first module may further include a second sensor, the second sensor being operable to measure a parameter of the environment in which the first module is to be deployed. In a particular embodiment, the output of the second sensor is used by the controller to determine the time at which a sensor element of the sensor array is activated. For example, the second sensor may be one of a pH sensor or a temperature sensor, as set out in relation to the second development.

In certain cases, the output of the sensor elements may depend on environmental conditions other than the concentration of analyte. For example, the output may depend on the pH and/or on the temperature. In that case, the output of the second sensor may be used to calibrate the output of the first sensor. Further features of this are set out with respect to the second development.

The first module may further include a third sensor, the third sensor being operable to measure a parameter of the environment in which the first module is to be deployed, different to the parameter measured by the second sensor. In a particular embodiment, the output of both the second and third sensors is used by the controller to determine the time at which a sensor element of the sensor array is activated. In an illustrative embodiment, the second and third sensors are selected from: a pH sensor, a temperature sensor, a dissolved oxygen sensor, a conductivity sensor, a biochemical sensor, an optical sensor and an acoustic sensor.

In a particular embodiment, a method of operating a sensing apparatus including a first module and a second module is provided. The first module includes a controller, a transmitter and an array of sensor elements. The method includes the controller activating at least one sensor element in the array independently of others in the array, so as to obtain a sensor output from the at least one sensor element at a first time t1. The method also includes the controller activating at least one further sensor element in the array independently of others in the array, so as to obtain a sensor output from the at least one further sensor element at time t2, different to t1. The method also includes transmitting sensor data from the first module to a receiver of the second module. Each sensor element is a biological sensor for detecting the presence of the same analyte in the environment in which the sensor array is to be deployed.

In a particular embodiment, the method further includes the controller activating the sensor elements sequentially at different times t so as to obtain a sequence of sensor outputs from the array, corresponding to the detection or absence of the analyte in the environment at the different times t.

In a particular embodiment, each sensor element is activated a maximum of one time only, to attempt to detect the presence of the analyte.

In a particular embodiment, a sensing device designed for passage through the digestive system of a human or animal body, or implantation into a human or animal body is disclosed. The device includes a first sensor for measuring a first parameter, electronic circuitry or software for calibrating the first sensor in accordance with a calibration routine and a transmitter for transmitting data derived from the first sensor's output to an external device.

The word “calibration” is used very generally here to indicate any or several of the following: assigning of a real physical value to the sensor output (e.g. assigning a pH, degrees Centigrade, oxygen concentration or other value to the voltage output by the sensor), adjusting or optimising the dynamic range of the sensor, forcing the sensor to give a zero output, making the sensor output relative to a known value and/or compensating for drift in the sensor.

In this way the sensor can be calibrated to give more accurate information or absolute values, or information particularly relevant to the user.

In a particular embodiment, a system for measuring a parameter is disclosed. The system including a first module and a second module including a receiver for receiving data transmitted by the first module's transmitter. The second module acts as the ‘external device’ previously mentioned.

In a particular embodiment, a system for measuring a parameter is disclosed. The system includes a first module in the form of a sensing device having a first sensor for measuring a first parameter and a transmitter for transmitting measurements made by said first sensor and calibration data generated by said first module to a second module. The system also includes the second module including a receiver for receiving data output by said first module's transmitter, and a processor for processing said data. The second module's processor is configured to calibrate the measurements made by the first sensor in accordance with a calibration routine and on the basis of calibration data sent by said first module. In a particular embodiment, the sensing device is a swallowable capsule or designed for implantation into a human or animal body.

The calibration routine may be a routine for optimising the dynamic range of the sensor. Here optimizing means improving and does not necessarily require the best possible dynamic range. The calibration routine may be a routine for compensating for drift of said first sensor output over time, the compensation being carried out in accordance with a model of sensor drift over time. The model of sensor drift over time may be a predetermined model stored in a memory. This predetermined model may be an empirically generated model or a theoretical model (if the physics of the sensor drift is well understood). In a particular embodiment, the model of sensor drift may be calculated while the sensor is in use, by extrapolating previous data points measured by the sensor. For example if there is a constant drift, then it is the discontinuities which are of interest. In this case a polynomial fit or moving average method can be used to model the drift in real time. In a particular embodiment, the sensor output is adjusted at regular intervals according to the model in order to compensate for sensor drift.

In a particular embodiment, a sensing device in the form of a swallowable capsule or a device designed for implantation into a human or animal body is provided. The sensing device includes a first sensor for measuring a first parameter, a second sensor for measuring a second parameter, a transmitter for transmitting data based on output from the first and/or the second sensor to an external device; and a controller for switching on the first sensor when the output from the second sensor displays a predetermined characteristic, or switching on the first sensor a set period of time after the output from the second sensor displays said predetermined characteristic.

In a particular embodiment, a sensing device in the form of a swallowable capsule is provided. The sensing device includes a first sensor for measuring a first parameter, a transmitter for transmitting data based on output from the first and/or a second sensor to an external device and a processor configured to detect a characteristic event in the first sensor output indicating that the sensing device is at a particular location in the body and to store in a memory and/or transmit to an external device, location data indicating the location of the sensing device.

A difficulty with swallowable capsules is that the dimensions of the capsule may be limited by the fact that it needs to be swallowable. Therefore the space inside the capsule and the number of components, which it can carry, may be limited.

In a particular embodiment a system having a first data sensing and transmitting module and a second receiving module which is configured to receive data from the first module and compensate it for drift due to variations in the power supply of the first module is disclosed. In this embodiment, the first module can be made very simple and even have a relatively inaccurate clock and/or fluctuating power supply because the second module is able to compensate for these shortcomings so that the user can still be provided with reasonably accurate data. Thus, the first module can be made smaller and with lower power consumption requirement without affecting the functionality of the capsule and the data integrity.

Particular embodiments disclosed may be useful for gathering data from a human or animal body. Additionally, particular embodiments may also have applications in the food and process control industries and, in fact, in any situation where a data sensing and transmitting device has to be kept small or light or to have minimal power consumption.

In a particular embodiment, an apparatus for gathering data is disclosed. The apparatus includes a first module having a first clock, at least one sensor, a power supply for supplying power to the first clock and the at least one sensor and a transmitter for transmitting sensor data from the at least one sensor. The apparatus also includes a second module having a second clock, a receiver and a processor configured to receive data sent from the first module's transmitter, estimate the first clock's clock rate and compensate the received sensor data for variations in the power of the first module's power source by adjusting the sensor data on the basis of the estimated first clock rate. The sensor data may be based on measurements made by the at least one sensor. In a particular embodiment, the first module is suitable for placement inside or passage through a human or animal body.

In a particular embodiment, the second module may compensate for drift in the sensor data from the first module due to variations in the power output from the first module's power supply. Usually variations in the power output from the power supply would cause corresponding variations in the values measured by the sensors (e.g. for some sensors and ADCs the output in response to a given stimulus will have a linear relationship with the power supplied by the power supply). Therefore, with the above apparatus it is possible to reduce or eliminate bulky (and power wasting) voltage regulating circuitry for regulating the voltage from the first module's power supply because the frequency, or clock rate, of the first clock is related to the voltage which it receives from the power supply. Therefore by estimating the clock rate of the first clock and noting its variations it is possible to compensate for (corresponding) variations in the sensor data. ‘Estimating’ the first clock's clock rate, includes noting the rate at which data is received by the second module and compensating the sensor data on the basis of the rate at which data is received. The rate at which data is received by the second module is in some transmission protocols relates directly to the first clock's clock rate.

In a particular embodiment, the first module's transmitter is a wireless transmitter and the second module's receiver is a wireless receiver. ‘Wireless’ means that the two are not connected together by a wire communication link. For example, the transmitter may be a radio transmitter and the receiver a radio receiver. Other possibilities include a magnetic induction, acoustic or optical communication link.

In a particular embodiment, the first module is a swallowable capsule. It may be designed for passage through the human digestive system, especially the gut. Typically it will be about the size of a large vitamin pill, but will usually not be larger than about 40 mm×12 mm.

In another particular embodiment, the capsule may be designed for passage through an animal's digestive system, especially but not limited to agricultural livestock such as cattle, sheep and pigs. In order that the capsule does not get stuck in the animal's stomach it may be less than about 50 mm long. In addition to mammals, particular embodiments may be used with non mammals, such as fish for fish farming.

In a particular embodiment, the first module may be an implant designed for implantation into the body, e.g., a human body. It may have an aperture for allowing the passage of body fluids past the module; e.g. it may be in the form of an annulus. In an illustrative embodiment, the first module is designed for insertion into the large bowel.

In a particular embodiment, the first module may be an implant designed for implantation into an animal body, e.g. it could be ‘stuck’ or placed in the animal's stomach. In this case it may typically be no more than about 13 cm long, e.g., about 12-13 cm for cattle and about 10 cm or less for a sheep.

Generally, the first module outputs a series of sensor values each corresponding to a sensor reading taken at a respective time, and for each respective sensor value, the second module's processor estimates the first clock's clock rate at the time when the sensor value was taken and adjusts each respective sensor value to compensate for variations in power from the first module's power source.

In a particular embodiment, the rate of the first clock is estimated on the basis of the time period, according to the second clock, which it takes for the second module to receive a predetermined amount of data, and the known number of clock cycles of the first clock which it takes for the first module to output the predetermined amount of data. The number of clock cycles can be known from the configuration or programming of a processor in the first module for outputting data and/or from the transmission protocol used by the first module. The predetermined amount of data may, for example, be a single bit of data, or a byte of data. To illustrate, if it is known that it takes x first clock cycles for the first module to transmit 1 byte of data and the second module receives 1 byte in t seconds, then the first clock rate is x/t Hz.

In a particular embodiment, the compensation is carried out on the basis of (i) a predetermined relationship between the sensor and the voltage supplied by the power supply and (ii) a predetermined relationship between the clock rate of the first clock and the voltage supplied to the first clock by the power supply. For example the sensor value readings taken by the sensor may be linearly related to the power source voltage at the time they were taken or the sensor data values may be linearly related to the voltage supplied by the power supply to the ADC or amplifier connected to the sensor. The power source voltage (V) may be related to the clock rate (f) of the first clock in accordance with an exponential, logarithmic or polynomial formula. Other possibilities will be apparent to a person skilled in the art. These predetermined relationships may be empirical or theoretical. The relationship between the power source voltage (V) and the first clock's clock rate in one embodiment is:

V=A log₁₀f+B

where A and B are constants.

In a particular embodiment, the sensor data is transmitted by the transmitter according to a protocol in which the data is split into one or more data packets, each data packet having a fixed predetermined length and wherein each data packet is separated from other data packets by a period of no signal transmission (‘zero-period’), having a fixed predetermined length. This makes it possible for the second module to easily distinguish the data packets from noise on the basis of the gaps (‘zero-periods’) between the data packets. For example, an iterative routine can search from both ends of the signal to find the data packets in-between the ‘zero’ periods in which no signal was transmitted. Preferably the length of the periods of no signal transmission is greater than the length of the data packet periods. In one embodiment the Manchester system is used as a communication protocol.

In a particular embodiment, each data packet has a start sequence of one or more bits marking the start of the data packet and a stop sequence of one or more bits marking the end of the data packet. This further helps identification of the data packets.

In a particular embodiment, the signal transmission from the first module to the second module is asynchronous. Here ‘asynchronous’ means that the signal transmission does not include data relating to the time at which the data was sent. Generally, the asynchronous transmission does not require a preliminary ‘handshaking’ step in which the two modules communicate with each other in order to synchronise and agree a transmission protocol. In a particular embodiment, the first module does not wait for the second module to confirm receipt of a data packet before sending the next data packet (e.g. as in a RS322 protocol). While this would be possible, it would require a receiver in the first module, which may increase the first module's size and power consumption.

In a particular embodiment, the at least one sensor is selected from a temperature sensor, a camera, a blood sensor, a pH sensor, a dissolved oxygen sensor, a conductivity sensor or a pressure sensor. Other possible sensors will be apparent to a person skilled in the art, on reading this disclosure. In a particular illustrative embodiment, the sensor is a sensor array as previously discussed.

In a particular embodiment, the first module does not have a regulator for regulating the voltage output from the first module's power supply. This saves power and is made possible because the second module is able to compensate for variations in the first module's power supply.

The first clock may have a low Q clock having a typical value of Q less than 20. The quality factor, Q, of an oscillator is defined as its resonance frequency divided by its resonance width. Generally speaking, the higher the Q, the purer its output frequency, since a high Q means that an oscillator will only output frequencies close to its natural resonance frequency. However, the system is able to cope even with a low Q resonator by using the central frequency. Furthermore, as a more accurate clock can be used in the second module to time stamp (assign a time to) the transmitted sensor data, the accuracy and stability requirements of the first module's clock can be further relaxed. Therefore, a small and cheap oscillator with low power consumption can be used, instead of a crystal oscillator, in order to regulate processing and transmission of data in the first module. For example, an RC relaxation oscillator, a ring oscillator, a bi-stable multivibrator, a Colpitts Oscillator or a Hartley Oscillator could be used. Other suitable low Q oscillators may be apparent to a person skilled in the art.

In a particular embodiment, the first module's transmitter transmits according to a CDMA system. This has several advantages, including the possibility of having several channels for communication with the second module (which acts as a base station). In a particular embodiment, there are a plurality of first modules, as described above, each first module transmitting on a different channel. Alternatively the plurality of first modules may communicate with the second module using frequency division multiplexing.

In a particular embodiment, the first module(s) may have a receiver for receiving a signal transmitted from a transmitter in the second module. In this case the communication link between the first and second modules may be half or full duplex. The presence of a receiver in each of a plurality of first modules makes it possible for communication to be carried out between the first modules and the second module according to a time division multiple access scheme.

In a particular embodiment, the second module's transmitter is a wireless transmitter and the second module's receiver is a wireless receiver. ‘Wireless’ means that the two are not connected together by a wire communication link (a wire link is possible, but not preferred). In an illustrative embodiment, the transmitter is a radio transmitter and the receiver a radio receiver. Other possibilities include a magnetic induction, acoustic or optical communication link.

In a particular embodiment, the processor is configured to pre-process the analogue signal from the receiver to generate a probability histogram to determine a voltage threshold to distinguish 0s and 1s in the analogue signal. In this way the thresholds for the 0s and 1s can be tailored to the operating conditions, accuracy can be improved and it becomes easier to detect even weak signals.

In a particular embodiment, the first module has its own processor and a memory. The memory may be a Read Only Memory, a read/writeable memory such as DRAM, SRAM or FLASH, or may include both types of memory. The read/writeable memory (if present) may be used for storing programme(s) for use on the processor, in this way the first modules operations are made flexible. The memory may also store instructions sent from the second module, or data relating to the sensed data etc.

Data transmitted from the first module to the second module, may be transmitted from the second module to another device for further analysis or display to a user. For example, the second module may be configured to transmit data to a mobile phone or other apparatus via Bluetooth or another protocol. The second module may be linked to a server, whereby data can be viewed and/or the second module operated remotely via the internet or another network. The data transmission between the modules and to any other devices and any access over a network may be made secure by encryption, private key and public key techniques or other secure protocols.

In a particular embodiment, a sensing device is provided in the form of a swallowable capsule or an implant for implantation into a human body. The sensing device includes a clock, at least one sensor, a power supply for supplying power to the clock and the at least one sensor and a transmitter for transmitting sensor data from the at least one sensor. In a particular embodiment the sensing device does not have a regulator for regulating the voltage output from the power supply and/or the sensing device is configured to transmit data to an external device in accordance with an asynchronous protocol. This configuration makes it possible to provide a compact sensing device with low power consumption and cheap components.

In a particular embodiment, the sensing device does not have a receiver for receiving data from an external device. This enables the sensing device to be kept compact and saves power consumption.

In a particular embodiment, the sensing device's clock is a low Q clock having a value of Q less than 20.

In a particular embodiment, the at least one sensor is a blood sensor. However, many other sensors, such as those previously discussed, may be used. In addition, the sensing device may have more than one sensor.

In a particular embodiment, the sensing device has an exterior casing with one or more grooves for channeling fluids towards one or more openings in the exterior casing. This feature may facilitate contact between the sensors and the environment which they are sensing.

In a particular embodiment, a swallowable capsule is provided. The swallowable capsule includes an exterior casing, at least one sensor and a transmitter for transmitting sensor data based on values measured by the at least one sensor. The exterior casing of the capsule has at least one helical groove, protrusion or indentation for causing the capsule to rotate as it passes through the intestinal tract. Rotation of the capsule means that its sensor(s) can gather data from all around the environment, not just one direction in which they are pointing, thus increasing the data available or reducing the number of sensors needed.

In a particular embodiment, a method of transmitting and receiving data is provided. The method may be performed in a system including a first module having a first clock, at least one sensor, a power supply for supplying power to the first clock and the at least one sensor and a transmitter for transmitting sensor data from the at least one sensor. The system may also include a second module including a second clock, a receiver and a processor. The method includes transmitting sensor data based on the output of the at least one sensor to the second module's receiver. The method also includes using the second module's processor to estimate the first clock's clock rate and compensating the received sensor data for variations in the power of the first module's power supply by adjusting the sensor data on the basis of the estimated first clock rate.

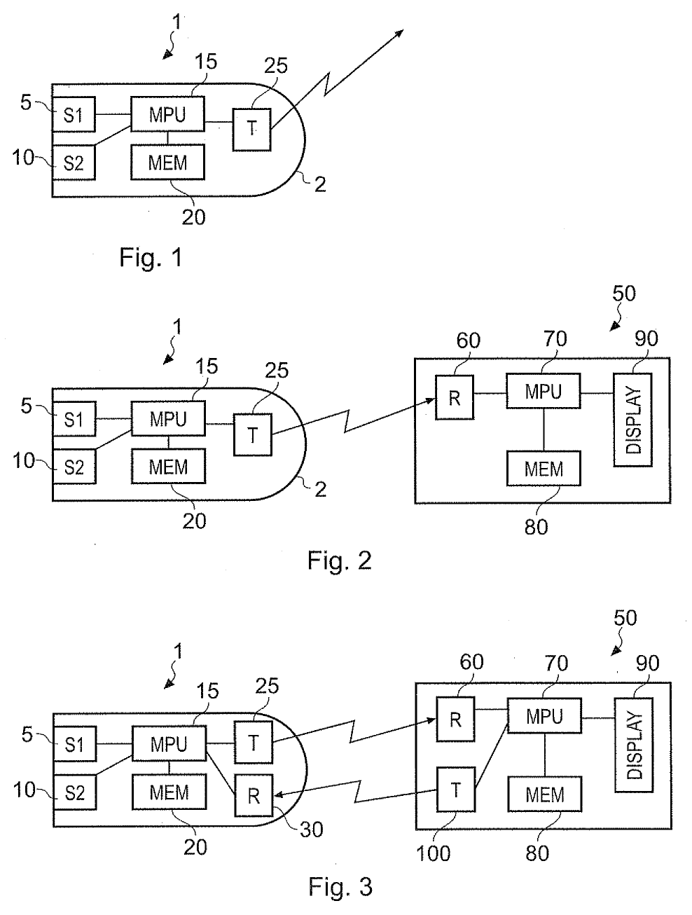

FIG. 1 shows a sensing device 1 in the form of a swallowable capsule. The capsule is designed so that it can be swallowed by a patient and passed through the gastro-intestinal tract. It is particularly useful for gathering data from the gastro-intestinal tract and bowels which may be used in the diagnosis of gastro-intestinal diseases. However, the capsule may also be used to gather data from other parts of the body, or from other environments.

The capsule has an exterior casing 2 which protects the internal electric components of the sensing device from liquids and acids in the body. The swallowable capsule is typically the size of a large vitamin pill, but in order to pass through the gut, it should be capable of leaving the stomach and therefore has a maximum size of approximately 40 mm×12 mm (for humans). If for use in an animal then the capsule should be no more than about 50 mm long, so as not to get stuck in the animal's stomach. The capsule and its components should preferably be made of materials which are safe for use in the human body, or animal body as the case may be, and approved by the relevant regulatory bodies (e.g. to an FDA or MHRA standard).

Particular embodiments also related to a sensing device designed for implantation in to a human or animal body. For example the sensing device could be designed for implantation in to one of the bowels, especially the lower bowel. In this case (for a human) it will have a typical size of less than about 40 mm×12 mm and may be in the form of an annular ring or other device with an aperture for allowing passage of body fluids. In other embodiments the sensing device may be an abdominal or thoracic implant device, with a typical size of less than about 100 mm×100 mm. If for an animal, it may e.g. be designed to be stuck or otherwise implanted or placed in the animal's stomach. In this case the device will typically be no more than about 13 cm long, e.g., about 12-13 cm for cattle, and about 10 cm or less for sheep. In all cases the implant device is preferably designed from suitable materials and according to the relevant standards.

The sensing device in the embodiment of FIG. 1 has a first sensor 5 for measuring a first physical parameter and a second sensor 10 for measuring a second physical parameter different to the first parameter. Typically the sensor will be exposed to the body by an aperture in the sensing device casing 2, or alternatively it may project from or be mounted on the exterior of the casing 2. The sensing devices may be selected from, for example, a pH sensor, a temperature sensor, a blood sensor, a dissolved oxygen sensor, a conductivity sensor, a biochemical sensor, or an acoustic sensor. This list is not limiting and other possibilities will be apparent to a person skilled in the art. While there are two sensors in the present embodiment, it would also be possible to have a sensing device with just one sensor or with three, four or even more sensors.

The sensing device 1 also includes a processor 15, memory 20 and transmitter 25. The first and second sensors 5, 10 are connected to the processor 15 which is configured to process data output from the sensors 5, 10 so that it can be transmitted to an external device via transmitter 25. The processor 15 is also configured to carry out calibration of the sensors 5, 10 as will be described in more detail later. The first memory 20 is connected to the processor 15 and used to store programs for running on the processor and calibration data generated by the processor. The processor 15 and memory 20 are preferably provided together on a single integrated chip designed by System-on-Chip (SoC design methodology). The sensors 5, 10 and transmitter 25 are provided on separate circuits and insulated from each other so as to minimize interference.

The transmitter 25 may be a wired transmitter or a wireless transmitter, such as a radio transmitter, or magnetic induction transmitter. It is configured to transmit data from the sensing device 1 to an external device and may use a standard protocol such as RS232 or a custom made protocol. The sensing device 1 also includes a power source, not shown in FIG. 1, in the form of one or more silver oxide batteries. In alternative embodiments, other batteries, or an induction loop powered by an external radio source could be used instead.

FIG. 2 shows a modular system for gathering data from the body. The system includes a first module 1 and a second module 50. The first module 1 is a swallowable capsule, as has already been described with reference to FIG. 1 and which has the same reference numerals. Alternatively the first module could be a sensing device designed for implantation into the human body as has already been discussed. The second module 50 is a base station. The base station includes a receiver 60 for receiving data transmitted from the first module 1, a second processor 70 for processing the received data, a second memory 80 for storing programs for execution on the second processor 70 and storing data and a display unit 90 for displaying data received and processed by the base station. The base station may take many forms. For example it may be a laptop computer, a PC or a custom made device. In the latter case it may be convenient for the base station to be worn around the waist of the user, for example on a belt. It is also possible for the system to have one or more intermediate modules between the sensing device and the base station 50. For example, there could be an intermediate module for receiving a signal transmitted by the sensing device's transmitter 25 and relaying the signal on to the base station 50. The intermediate device may or may not carry out processing of the data. It may be conveniently provided in a belt or other item which can be worn by the patient.

FIG. 3 shows another example of a modular system having a first module 1 and a second module 50. The first module 1 and the second module 50 are similar to the first and second modules illustrated in FIG. 2 and like parts are have like reference numerals. Therefore only the differences will now be described. Whereas in FIG. 2 there was a one-way communication link between the sensing device 1 and the base station 50, i.e. transmission from the sensing device to the base station, in the system of FIG. 3 communication is possible in both directions. The sensing device 1 has both a transmitter 25 and a receiver 30. Likewise, the base station 50 has both a receiver 60 and a transmitter 100. In this way data can be sent from the sensing device 1 to the base station 50 via the transmitter 25 and receiver 60. Data and/or instructions can also be sent from the base station 50 to the sensing device 1 via the base station transmitter 100 and sensing device receiver 30. While the sensing device's transmitter 25 and receiver 30 have been shown as separate components in FIG. 3, they may also be provided as a single component, e.g. a transceiver. The same is true of the base station's receiver 60 and transmitter 100. The two-way communication may be via a half or full duplex link. In the system of FIG. 3 as for the system of FIG. 2, there may be one or more intermediate modules for relaying signals between the sensing device 1 and base station 50.

An important consideration for both swallowable capsule and implant sensing devices is to reduce or minimize the power demands from the electric components. The amount of available power may be limited by the size of the device, especially where the sensing device is a swallowable capsule or design for implantation into a small part of the body. Furthermore, where the power supply is provided by a battery, then it may not be possible to re-charge the battery until the sensing device is removed from or passes out of the body. In the case of a swallowable capsule, the power supply should last for as long as 19 hours and not all of the measurements taken in this time will be of interest. For example, if the sensing device is being used to gather data from the large bowel, then readings taken while the capsule is in the small bowel will not be of interest.

Therefore, the sensing device 1 is configured so that the first sensor 5 can be activated by the second sensor 10. The processor 15 may act as a controller to turn on the first sensing device 5 when certain characteristics are detected in the output from the second sensor device 10. These characteristics and the method of detecting them are stored on the memory 20.

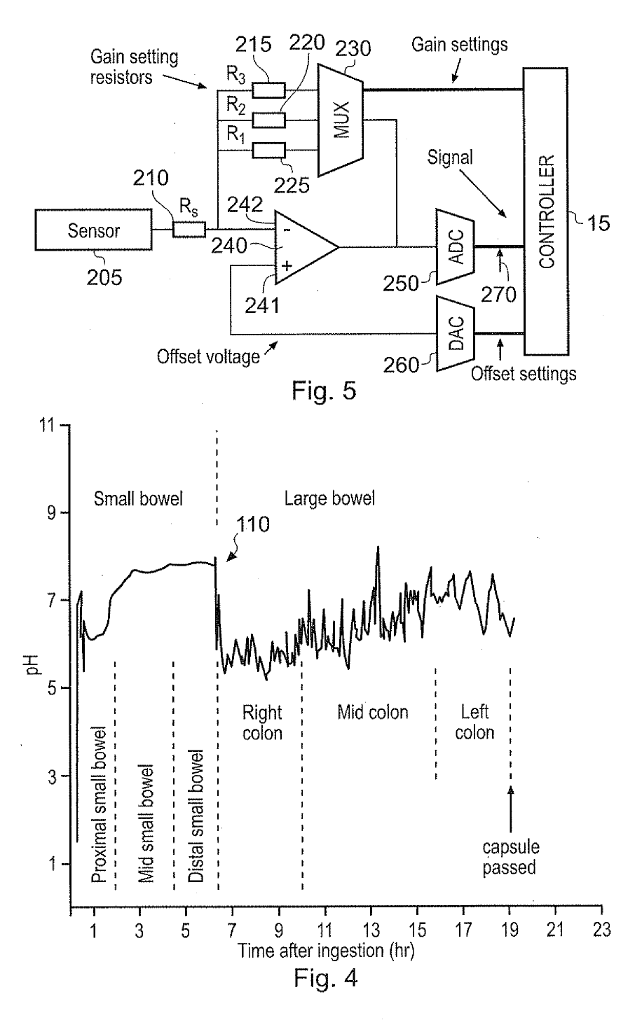

An example will now be given where the first sensor 5 is a blood sensor, more particularly a Faecal Occult Blood (FOB) sensor and the second sensor 10 is a pH sensor. FIG. 4 shows the pH detected by the second sensor as it passes through a human digestive system. It can be seen that there is a characteristic drop in pH 110 when the sensing device passes from the small bowel to the large bowel. In the small bowel the pH is above 7 and slightly alkaline, but immediately after entry into the large bowel the pH is below 7 and mildly acidic. The processor 15 detects this characteristic steep drop in output from the second sensor 10 (measuring pH) and accordingly switches on the first sensor 5. In this way power is saved, as the first sensor is switched off for the first six to seven hours of operation.

This principle is not limited to a pH sensor regulating the switching on and off of a blood sensor. It can be used in any other situation where it is desirable to activate a first sensor on the basis of the output of a second sensor. Other applications will be apparent to a person skilled in the art. In this way power can be saved because one of the sensors can be turned off for at least some of the time. This technique is especially valuable where the first sensor requires a lot of power to operate, but the second sensor requires a relatively small amount of power. The technique can also be used where the first sensor has a short operational lifetime, as it can then be switched on only when it is needed.

In the above example, a memory 20 contained a program to enable the processor 15 to detect a characteristic in the output of the second sensor. This technique is the one which is used in the embodiments of FIGS. 1 and 2 where there is a one-way communication link between the sensing device 1 and the base station 50, such that the sensing device 1 can only transmit data. The embodiment of FIG. 3 can also have a program on the memory 20 to enable the sensing device 1 to autonomously switch on and off the first sensor on the basis of the output of the second sensor. However, because the sensing device of FIG. 3 also has a receiver, an alternative implementation is possible. In this alternative implementation the sensing device processor 15 controls transmission of data from the first and second sensors to the base station 50. The processor 70 on the base station 50 then processes this data, stores in memory 80 and optionally displays on display 90. The processor 70 can be configured to detect a characteristic in the output from the second sensor and in response to detecting this characteristic, sends an instruction to the sensing device processor 15 (via base station transmitter 100 and sensing device receiver 30). This instruction instructs the processor 15 to switch on the first sensor 5. In other words, the processor 15 controls switching on and off of the first sensor 5 in accordance with an instruction from the base station 50. Furthermore, as an alternative or in addition to the processor 70 of the base station 50 detecting the characteristic, it would be possible for a user of the base station 50 to directly instruct switching on or off of the first sensor by inputting a command to the base station 50. The user may do this in response to data displayed on the base station display 90. It is also possible for the control program or a user switch the first sensor 5 on once a set period after the characteristic event has elapsed.

Not only can a characteristic event in the output from a sensor be used to control a switching on or off of a second sensor, it can also be used to determine the location of the sensing device. This is particularly useful where the sensing device is a swallowable capsule which passes through the body. In order to implement this, the microprocessor 15 is configured to detect a characteristic event from either the first or second sensor 5, 10, which indicates the location of the sensing device 1. For example, as explained above with reference to FIG. 4, a characteristic change in the pH from alkaline to acidic indicates that the capsule has left the small bowel and entered the large bowel. This principle is not limited to pH and other parameters can be used to indicate the location of the sensing device 1. The characteristic indicative of the location of the device may be where the output of a sensor passes a predetermined threshold, rises and falls in a characteristic manner or undergoes another recognizable pattern.

The way in which the sensors 5, 10 of the sensing device 1 may be calibrated will now be described. In this specification calibration is used in a general sense to mean optimization of the dynamic range of the sensor, assigning actual parameter values to the sensor output, compensating for drift in the sensor output, auto-zeroing the sensor output and/or referencing the sensor output to a desired known value. Any or all of these calibration techniques may be used, either at the same time or at different points in the life of the sensor. Each technique will now be described in turn.

The first calibration technique is adjusting the dynamic range of the sensor. The dynamic range of the sensor is the range of actual values that it is able to accurately measure. For example, a temperature sensor capable of measuring temperatures anywhere in the region 0 to 100° C., but which becomes inaccurate below 0 and above 100° has a dynamic range of 0 to 100° C. It is desirable to adjust the dynamic range in order to improve or optimize the range of values which can accurately be measured and so that the dynamic range corresponds to the conditions which the sensor is likely to be exposed to. The dynamic range of the sensor is controlled by analogue circuitry connected to the sensor. For example, an offset voltage applied to the sensor can be adjusted. Alternatively, where the sensor is connected to an amplifier the offset voltage applied to the amplifier or the gain of the amplifier can be varied in order to adjust the dynamic range of the sensor. In some cases the sensor itself will be an amplifier (e.g. ISFETs are sometimes used as pH sensors) and in this case the gain or offset voltage of the sensor itself can be adjusted. Some sensors which are not amplifiers, also have an offset voltage and this can be adjusted in order to achieve the same effect.

FIG. 5 is a diagram showing circuitry for controlling the dynamic range of a sensor 205 (the same scheme may be used for any other sensors on the sensing device). The sensor 205 outputs an analogue voltage in response to a physical stimulus which it is exposed to (e.g. the ambient environment or a substance which it is exposed to). This analogue voltage passes through the sensor resistor 210 to a variable amplifier 240. The variable amplifier 240 amplifies this signal and outputs the amplified signal to an ADC 250. The ADC converts the analogue signal into a digital signal which it inputs to controller 15. In this embodiment controller 15 is the same as processor 15 in FIG. 1, but in alternative embodiments it may be a separate chip which is connected to the sensing device's processor. The gain of the variable gain amplifier 240 and the offset voltage applied to the amplifier 240 is controlled by the controller 15. The offset voltage is controlled by the controller outputting a digital signal indicating a desired offset setting to DAC 260. The DAC 260 converts the digital signal to an analogue voltage which is input as an offset voltage to terminal 241 of variable amplifier 240. The gain of the variable amplifier is controlled by the controller 15 outputting a control signal containing gain settings to multiplexer 230, which then applies voltages corresponding to these gain setting resistors 215, 220 and 225, which results in the gain setting signal being input to terminal 242 of variable gain amplifier 240. The effected output of the sensor to the processor 15 is the output 270 from ADC 250.

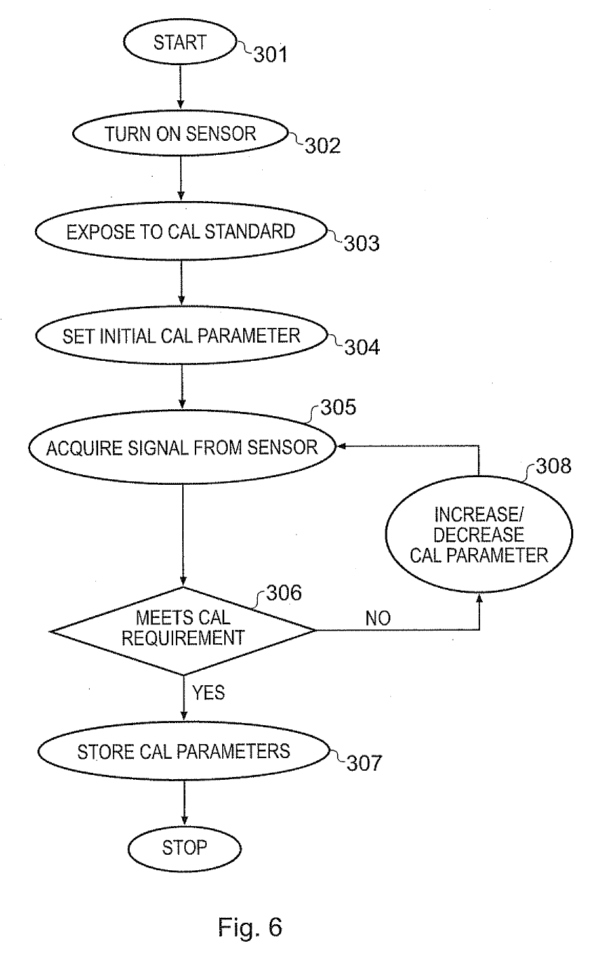

A calibration routine for adjusting the dynamic range of the sensor 205 (or any of the other sensors) will now be described with reference to FIG. 6. The calibration routine is started in step 301. Usually the calibration routine for adjusting or optimizing the dynamic range of the sensor 205 will be carried out when the sensing device 1 is first switched on. The sensing device 1 can conveniently be switched on by activating a magnetic switch inside the device. In step 302 the sensor 205, whose dynamic range is being adjusted, is switched on. In step 303 the sensor 205 is exposed to a calibration standard (i.e. a known stimulus). The calibration standard may be a reference voltage, a known response when the device is dry (i.e. in air) or a known substance. A preferred technique is that the sensing device 1 is sold in a package filled with calibration fluid and that the calibration is activated (e.g. by magnetically switching on the device) prior to breaking the package seal. In this way the calibration can be carried out under very controlled conditions with no inconvenience to the user.

In step 304, the initial calibration parameter is set. This calibration parameter relates to either the gain or offset voltage to be applied to the amplifier 240 (or to the sensor 205 itself in alternative embodiments). The initial calibration parameter may be a value stored in the memory 20 of the sensing device 1.

In step 305 an output signal 270 is acquired from the sensor 205. In step 206 the controller 15 compares the acquired output signal 270 of the sensor 205 with a calibration requirement. The calibration requirement is a desired value for the output of the sensor 205. The calibration requirement may be stored in the memory 20 of the sensing device 1. It may be a value which is selected in order to give a desired (e.g. optimal) dynamic range for the sensor 205. For example, if the sensor 205 is a pH sensor, the calibration standard is a reagent having a pH 7 and the amplifier 240 has an output range 0-12 mV, then the calibration standard may be set to 7 mV. This will give a large dynamic range to the sensor. If, however, the sensor output 270 in response to a pH of 7 was 11 mV, then the dynamic range of the sensor 205 will be compromised. In that case the amplifier 240 would become saturated and output its maximum voltage of 12 mV at pH 8 or so and the dynamic range would have an upper limit of pH8.

If at step 306 the output signal 270 of the sensor 205 meets the calibration requirement, then the calibration parameters are stored in the memory 20 of the first sensing device 1 and optionally also transmitted to base station 50. If the sensor output 270 in step 306 does not meet the calibration requirement, then the controller 15 increases or decreases the calibration parameter accordingly by varying the gain setting or offset setting of the amplifier 240. The output signal 270 is then checked again and step 206 repeated as often as necessary until the calibration requirement is met. Once the calibration requirement is met then the routine proceeds on to step 307 which is described above.

In the above description the calibration routine of FIG. 6 is carried out autonomously by the sensing device 1. That is the calibration routine is stored on the memory 20 and carried out by the sensing device processor 15. If the sensing device has a receiver, as in embodiment of FIG. 3, then it is possible for the calibration routine to be carried out partly at the base station 50. In that case the controller 15 simply forwards the sensor output to the base station 50 and controls the calibration parameters (gain and offset) in response to instructions sent by the base station 50. The initial calibration parameter at step 304 and assessment as to whether the sensor output meets the calibration requirement at step 306 can all be carried out by the base station processor 70. The base station processor 70 can also instruct the sensing device's processor 15 to increase or decrease the calibration parameters at step 308 if necessary.

Whether the adjustment of the sensor's dynamic range is carried out autonomously by the sensing device 1 or in collaboration with the base station 50, it is desirable for the system device 1 to forward the final calibration parameters to the base station as calibration data.

The sensor output after conversion by the ADC is in digital form and usually will be a series of numbers relating to the voltage output by the sensor. At some point this sensor data may be converted into actual physical values representing the measured parameter (e.g. pH, degrees centigrade, oxygen concentration etc depending on the type of sensor). This calibration routine to assign actual values to the sensor data may conveniently be carried out at the same time as the routine of FIG. 6 for adjusting the dynamic range of the sensor. However, these two routines do not depend upon each other and may be carried out independently. It is quite possible to have a system in which the dynamic range of the sensor is optimized, but actual values are never assigned to the sensor data (so only relative change and not absolute values are measured). It is also quite possible to have a system in which no adjustment of the sensor's dynamic range is ever carried out, but in which absolute values are assigned to the sensor data. Additionally, both of these calibration functions may be carried out so that the system provides absolute physical values over an optimum dynamic range.

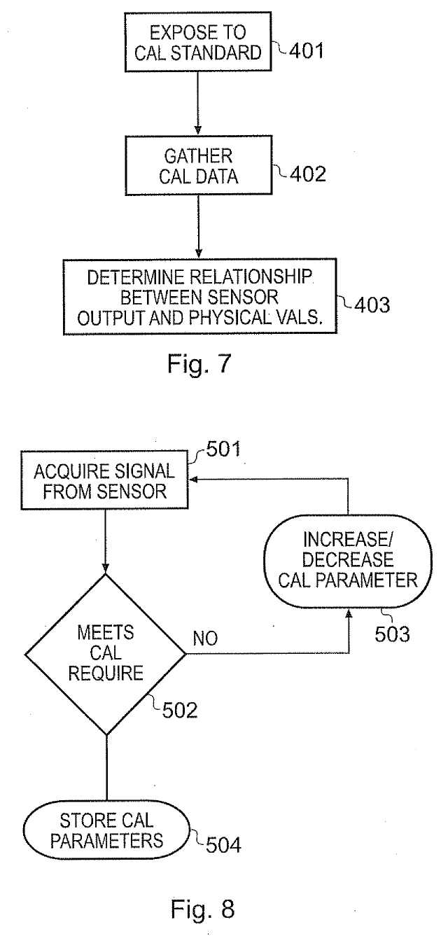

FIG. 7 shows a calibration routine for assigning actual physical values to the sensor data. In step 401 the sensor is exposed to a calibration standard as described for step 303 in the routine of FIG. 6. In fact this step may conveniently be carried out at the same time as step 303 of FIG. 6. Next, calibration data, including at least the data output by the sensor in response to the calibration standard, is collected. Where this routine is carried out at the same time as the FIG. 6 routine for adjusting the dynamic range, the sensor calibration data should be collected after the dynamic range has been finally adjusted and may take the form of a flag simply confirming that the calibration requirement has been met. In step 403 a relationship between the sensor output in response to the calibration standard and an actual physical value is determined by a processor. For example, if the sensor being calibrated is a temperature sensor, the calibration standard is 30° C. and the output of the sensor in response to the calibration standard is 300 mV, then the processor may determine that the sensor output can be divided by 10 in order to give the temperature in ° C. In other cases, especially where the relationship is non-linear, a more complicated relationship will have to be determined and it may be necessary to take more than one set of calibration data.

In general it is most efficient for the assignment of actual values to the sensor output to be carried out at the base station 50. Therefore, only steps 401 and 402 may be carried out by the sensing device 1 and step 403 may be carried out at the base station. In this case, the base station 50 can instruct gathering of the calibration data by sensing device processor 15 in step 402. If the base station 50 already knows the calibration standard and the calibration requirement then the calibration data may simply be a flag sent from the sensing device 1 indicating that the calibration requirement has been met. In other cases, it may be necessary for the sensing device 1 to forward data relating to both the calibration standard and the actual output of the sensor in response to the calibration standard. In other cases it may be possible for the processor 70 on the base station 50 to work out the relationship on the basis of calibration parameters (e.g. gain and offset) stored in the memory 20 of the sensing device 1 and transmitted to the base station 50 and the calibration standard (i.e. the absolute value of the known stimuli, such as pH 8, 30° C. for example).

It is also possible for the calibration relating the sensor output to actual physical values to be carried out entirely on the sensing device 1. In this case the sensing device 1 can be configured to determine the relationship in step 403 and then convert all of the sensor output into actual physical values for encoding and transmission to the base station in accordance with the transmission protocol. However, this approach puts a fairly heavy load on the sensing device's processor.

An auto-zero calibration routine will now be described, with reference to FIG. 8. It is sometimes desirable to force the sensor to return a null response (or approaching zero output). This may be used, for example, when it is desired to measure relative changes in a physical parameter, rather than absolute values. In this case maximum sensitivity can be achieved by auto zeroing the sensor. This will typically be carried out when the sensing device 1 has reached a site of interest. The auto-zero routine may be controlled by the processor 15 of the sensing device 1, autonomously in accordance with instructions 20 stored in its memory 20. Alternatively, where the sensing device 1 has a receiver, as in the embodiment of FIG. 3, the calibration routine may be carried out by the processor 15 of the sensing device 1 in accordance with instructions issued by the processor 70 of the base station 50.

In step 501 of the auto-zero routine, the output signal from the sensor being calibrated is acquired. In step 502 the processor checks whether the acquired signal meets the calibration requirement, which for the auto-zero is 0 or approaching 0. If the output meets this calibration requirement then the calibration parameters (gain and/or offset) are stored in memory 20 of the sensing device 1 and optionally also transmitted to the base station 50. If the calibration requirement is not met then the calibration parameter (gain or offset of the amplifier or sensor) is increased or decreased in step 503 and checked again in steps 501 and 502. This process is repeated until the output from the sensor is 0 or approaching 0 meeting the calibration requirement. Once the calibration requirement is met then the calibration parameters are stored in step 504 as described above.

The routine of FIG. 8 may alternately be used to force the sensor to give an output which is relative to a desired value. For example, if it is known that the body part being monitored should have a pH of 6 then the calibration requirement can be set such that all the sensor outputs are relative to pH 6. This is similar to auto-zeroing to pH 6, except that in the auto-zero routine the sensor is forced to output 0 in response to the environment it is currently in, whereas in this implementation the sensor need never have been exposed to pH 6 and the calibration requirement is a nominal one calculated on the basis of an expected output at pH 6.

Finally, it may be desirable to calibrate the sensor to compensate for drift. It has been found that many sensors suffer a drift in their output voltage over time, even when exposed to constant conditions. This is often the case in a sensor which comes into physical contact with the substance they are detecting, due to ions from the substance entering into the sensor and remaining there even after the substance has moved away.

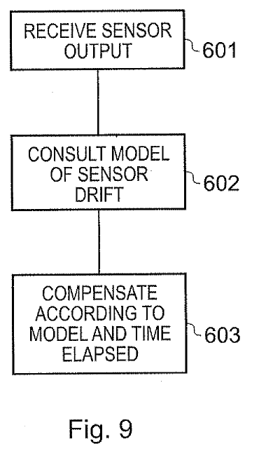

A calibration routine for compensating for sensor drift over time is shown in FIG. 9. The base station processor 70 receives sensor data transmitted from the sensing device 1 in step 601. It then consults a model of sensor drift in step 602. This model is stored in the base station's memory 80. This model may be a model based on empirical data relating to the drift of that type of sensor over time. Alternatively it may be a theoretical model of sensor drift based on a theoretical model of sensor drift for that type of sensor. Alternatively, the model may not be a predetermined model stored in the base station's memory, but may be a model of sensor drift which is calculated in real time on the basis of previous readings returned by the sensor. For example, a moving average method or a polynomial fit can be used to model the drift in real time. In this case the model will change as the data from the sensor changes. A suitable polynomial method is described in Irvine et al, Variable-Rate Data Sampling for Low-Power Microsystems using Modified Adams Methods, IEEE Transactions on Signal Processing, Vol 51, No 12, December 2003. In that paper the method is described in the context of power saving in a sensing device by controlling the sample rate to reflect the rate of change of the sampled data, however the same mathematical technique can also be applied to model sensor drift. The drift compensation is typically best carried out by the base station processor 70. However, it is possible to carry out the compensation by the base station processor in collaboration with the sensing device processor or by the sensing device processor autonomously on its own. Where part or all of the compensation is carried out on the sensing device, this may be by varying of the gain and offset voltage of the sensor or an amplifier connected to the sensor.

A study on modeling of drift in ISFET pH sensors will now be discussed in detail. The ISFETs in this study had large, negative threshold voltages of approximately −5V. In general ISFETs may have a range of large threshold voltages for their CMOS ISFETs. The floating-electrode ISFET has a similar structure to an EPROM2 device, which uses charge trapped on the floating gate of a transistor to store a ‘1’ or a ‘0’ in memory. These chips have a quartz window in the package that allows them to be erased by exposure to ultraviolet (UV) radiation.

The UV light excites the electrons on the gate to such an extent that they can escape over the oxide energy barrier and discharge the gate. UV radiation has been shown to be an effective way of increasing the CMOS ISFET threshold voltage towards standard p-type MOSFET values (−0.7V).

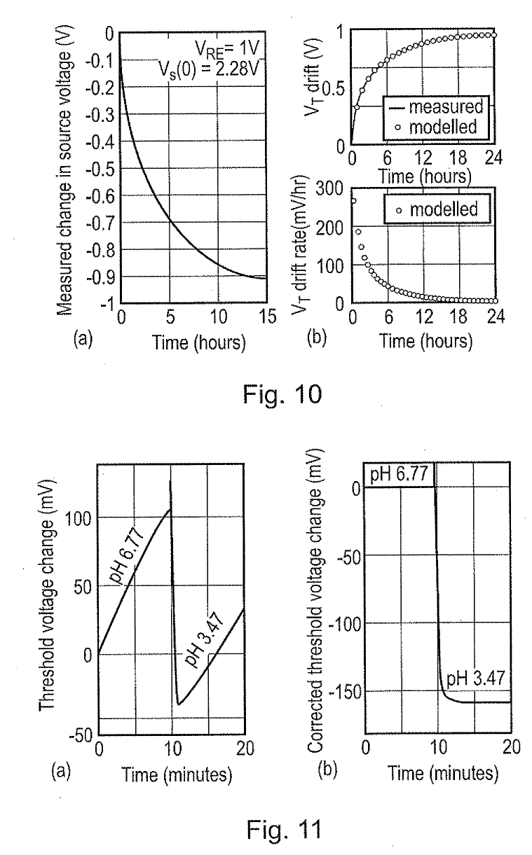

The ISFETs also displayed significant threshold voltage drift under constant bias conditions. This can be seen in FIG. 10(a), which shows that source voltage drops by 900 mV over a period of 15 h. Threshold voltage drift for non-CMOS silicon nitride ISFETs has been successfully modeled by a ‘stretched exponential’ time dependence. Upon exposure to an aqueous solution, silicon nitride is known to form a thin, hydrated surface layer as hydrogen ions diffuse into the material. The growth of a modified surface layer affects the overall insulator capacitance, which in turn influences the threshold voltage. In amorphous silicon, the surface layer is shown to grow by a mechanism known as ‘dispersive transport’ and its thickness follows a stretched-exponential time dependence. It is reasonable to assume that surface layers for other glassy materials, such as silicon nitride, will grow in the same manner. Since the layer thickness has a stretched-exponential time dependence, so too will the threshold voltage drift:

VT(t)=VT(∞){1−exp_{—}−t/τ)β (Equation 1)

where VT(∞) is the ultimate change in threshold voltage as a result of drift, τ the time constant, and β the dispersion parameter, characterizing the dispersive transport of hydrogen. A non-linear curve-fitting algorithm (Levenberg-Marquardt) was used to fit the parameters [VT(∞), τ, β] in Equation 1 to the measured values of VT(t) (equal to −□VS(t)). The values calculated by this method were:

VT(∞)=963 mV, τ=3.48 h, β=0.722

The curves in FIG. 10(b) show that a modeled drift rate of less 123 than 5 mV/h will be achieved after 18 h in solution and under bias. In contrast, the values extracted for the non-CMOS silicon nitride ISFET in another study were:

VT(∞)=79.7 mV, τ=53.4 h, β=0.613.

In that study, the ultimate drift VT(∞) was 12 times smaller, and the time constant τ was 15 times longer than were measured here. The smaller drift and larger time constant may be explained in terms of the deposition method used to form the nitride layer. That study used low-pressure chemical vapour deposition (LPCVD), which is a high temperature (700-800° C.) method resulting in a dense film with few pinholes. The nitride passivation layer used in aCMOS process is deposited after the metal layers, so a low-temperature (250-350° C.) plasma-enhanced CVD (PECVD) process should be used. Films deposited by PECVD have a lower density and contain pinholes. This would allow more hydrogen to diffuse into the nitride more quickly, and could explain the much larger drift and smaller time constant measured in this study.

The same curve-fitting technique was used to remove the drift from the pH sensitivity measurement in FIG. 11. The threshold voltage changes by approximately −159 mV for a change in pH of −3.3 units, giving a sensitivity of 48 mV/pH. FIG. 11(a) is a graph of ISFET threshold voltage response to a change in solution pH as measured and FIG. 11(b) shows the response with drift correction applied.

The skilled person will appreciate that the calibration routines and schemes applied to the pH sensor described above will also be applicable to other forms of sensors. In particular, similar calibration routines and schemes can be applied to a sensor consisting of an array of sensor elements, for example an array of sensor elements capable of sensing FOB, as described in more detail below. In a preferred embodiment, each element of such an array is a one-shot sensor. Thus, the calibration can operate so that the output of one sensor element can be used to calibrate the output of another sensor element in the array. Also, the output of a different type of sensor (e.g. pH or temperature sensor) can be used to calibrate the output of one or more of the sensor elements.

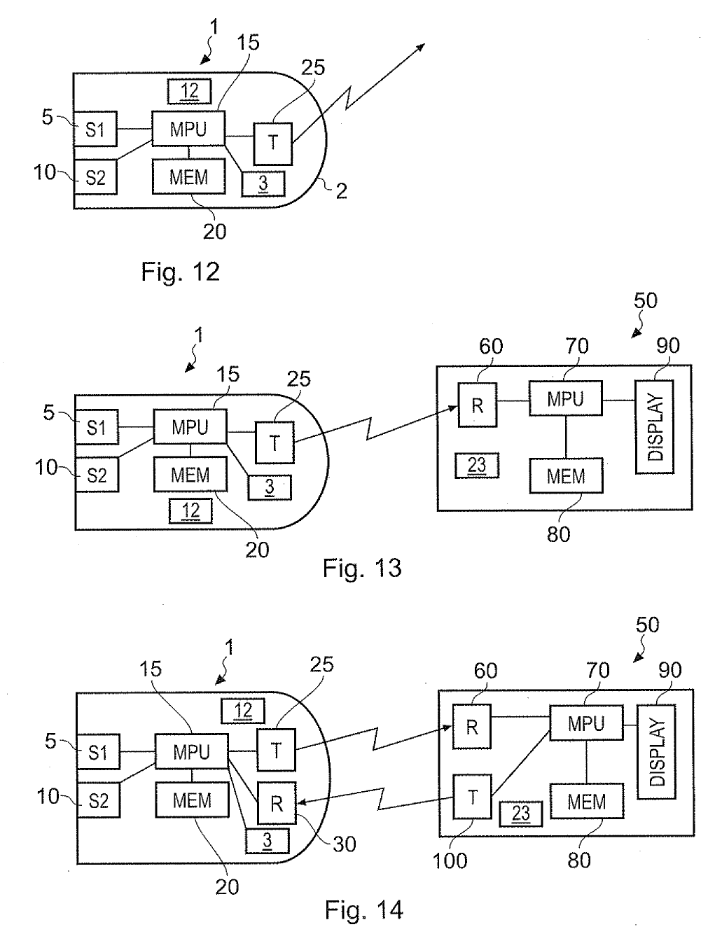

FIGS. 12 to 14 show a modification of the devices and systems of FIGS. 1 to 3. For this reason, similar reference numbers are used. Only the additional features will be described in detail below.

In each of FIGS. 12 to 14, memory 20 includes both a ROM and a rewritable memory (e.g. EPROM); the re-writable memory stores programs for running in the processor 15 and data generated by the processor. As the memory has a rewritable portion the sensing device can be reprogrammed after manufacture and even during operation.

The sensing device 1 also includes a power supply 12 for supplying power to the various components of the sensing device and a first clock 3 for regulating operation of the processor 15. The power supply is in the form of one or more silver oxide batteries. In alternative embodiments, other batteries, or an induction loop powered by an external radio source could be used instead.

FIG. 13 shows a modular system for gathering data. The system includes a first module 1 and a second module 50. The first module 1 is a swallowable capsule, as has already been described with reference to FIG. 12 and which has the same reference numerals. Alternatively the first module could be a sensing device designed for implantation into the human or animal body as has already been discussed. In still further embodiments the sensing device may be any device having a sensor and linked to a second module, it need not be a swallowable capsule or a body implant. For example, the sensing device may be for topical application, e.g. in a wound dressing.

The second module 50 is a base station. The base station includes a receiver 60 for receiving data transmitted from the first module 1, a second processor 70 for processing the received data, a second clock 23, a second memory 80 for storing programs for execution on the second processor 70 and storing data, and a display unit 90 for displaying data received and processed by the base station. The base station may take many forms. For example it may be a laptop computer, a PC or a custom made device. In the latter case it may be convenient for the base station to be worn around the waist of the user, for example on a belt. The second clock 23 is preferably an accurate clock such as a crystal oscillator. It is used to regulate the second processor 70 and to time stamp data received from the first module, as will be discussed in more detail later. These two functions may optionally be carried out by two separate clocks in the second module.

Although not shown in FIGS. 13 and 14, it is possible for the system to have one or more intermediate modules between the sensing device 1 and the base station 50. For example, there could be an intermediate module for receiving a signal transmitted by the sensing device's transmitter 25 and relaying the signal on to the base station 50. The intermediate device may or may not carry out processing of the data. It may be conveniently provided in a belt or other item which can be worn by the patient. FIG. 15 shows examples of various configurations of first and second modules which can be used with embodiments disclosed herein. In FIG. 15(a) a small (S) first module 1 is linked to a large (L) second module 50. There is just one first module and one second module as shown in FIGS. 13 and 14. In FIG. 15(b) there are a plurality of first modules 1a to 1f, each of which communicates with a second module 50 which acts as a base station. This can be achieved, for example, by splitting the communication bandwidth into a plurality of channels using a scheme such as CDMA, or TDMA etc. For TDMA to work the first modules 1a to if may have a receiver for receiving signals sent from the second module 50 (as in FIG. 14). In FIG. 15(c) first modules 1a to 1c have a communication link to an intermediate module 7a. The intermediate module 7a has a communication link to large second module 50. The intermediate module 7a is configured to receive signals from first modules 1a to 1c and relay the signals to the second module 50 which acts as a base station. First modules 1d to 1f have a communication link to an intermediate module 7b which also relays signals to the base station 50.

In alternative embodiments, it would be possible for the module 7a to be the base station and for the large module 50 to be a remote device for storing and/or carrying out further processing of data sent from base station 7a or 7b. In that case the remote device 50 may be a computer or storage facility linked to module 7a and 7b over a computer network or the internet, for example.

In order for the power demands of the sensing device to be limited, the power supply circuitry of the sensing device 1 may be kept simple and may not include a voltage regulator. As there is no voltage regulator, space is saved and power consumption is reduced. Furthermore the first clock 3 of the sensing device 1 (herein after also referred to as the first module) is a RC relaxation oscillator. Other possible alternatives for the first clock 3 include an astable oscillator, a multi vibrator, a Coll-Pitts oscillator or a Hartley oscillator. These clocks are smaller, cheaper and consume less power than the conventionally used crystal oscillator. Other possibilities may be apparent to a person skilled in the art. The aforementioned clocks, other then the crystal oscillator, have a low Q. However, even with a Q of 10 to 20, the system is still able to operate as the central frequency is easily discernable. A clock with Q in the range 2-10 may also be possible. In this embodiment the first clock 3 is provided on the same integrated chip as the processor 15 and memory 20, in order to save space. However, it would be possible to have it mounted on a separate chip or circuit board.

As the voltage supply is not regulated, its output voltage is not stable. It will vary over time (e.g. as the batteries run down) and in response to changes in ambient conditions (e.g. temperature). The electronic components of the first module will be affected by variations in the power supply voltage. For example, all of the sensors will be coupled to the processor 15 by an ADC. The response of the ADC varies dependent upon the power supply voltage (usually in a linear fashion). Some sensors will themselves also vary in response, dependent upon the voltage which they are supplied from the power supply (e.g. the output of many temperature sensors varies linearly with the power supply voltage at constant temperature). Therefore the sensor data transmitted to the second module will not be an entirely accurate reflection of the values measured by the sensors, because it will be corrupted by variations due to the power supply voltage. The second module is able to compensate for these variations because the frequency (clock rate) of the first module's first clock 3 also varies according to the power supply voltage.

Thus, where the base station 50 (the second module) is able to detect or estimate the frequency of the first clock 3 at the time at which each sensor value or set of sensor values was taken by the sensors 5, 10 then it can compensate these sensor values accordingly.

The compensation can be carried out by first determining the first clock frequency for each portion of sensor data (i.e. for each sensor value or set of sensor values). Methods of estimating the first clock frequency are explained later. From the first clock frequency it is possible to calculate the voltage supplied by the power supply on the basis of a predetermined relationship between the power supply voltage and the first clock frequency. This predetermined relationship may be calculated empirically or theoretically and in the case of certain clocks may be specified by the manufacturer. In one experiment it was found that the power supply voltage (V) exhibited a logarithmic dependence upon the first clock frequency (f). This could be expressed in the formula V=A log₁₀f+B, where A and B were constants. This is just given by way of example and other clocks may exhibit logarithmic dependencies, or exponential or polynomial dependencies. Once the power supply voltage has been calculated, the compensation can be carried out on the basis of a predetermined relationship between the power supply voltage and the sensor values in the sensor data transmitted to the base station 50. This predetermined relationship may be calculated theoretically or empirically. In most cases it will be a linear relationship as the first module's ADC will usually have an output which varies linearly in response to changes in the power supply voltage.

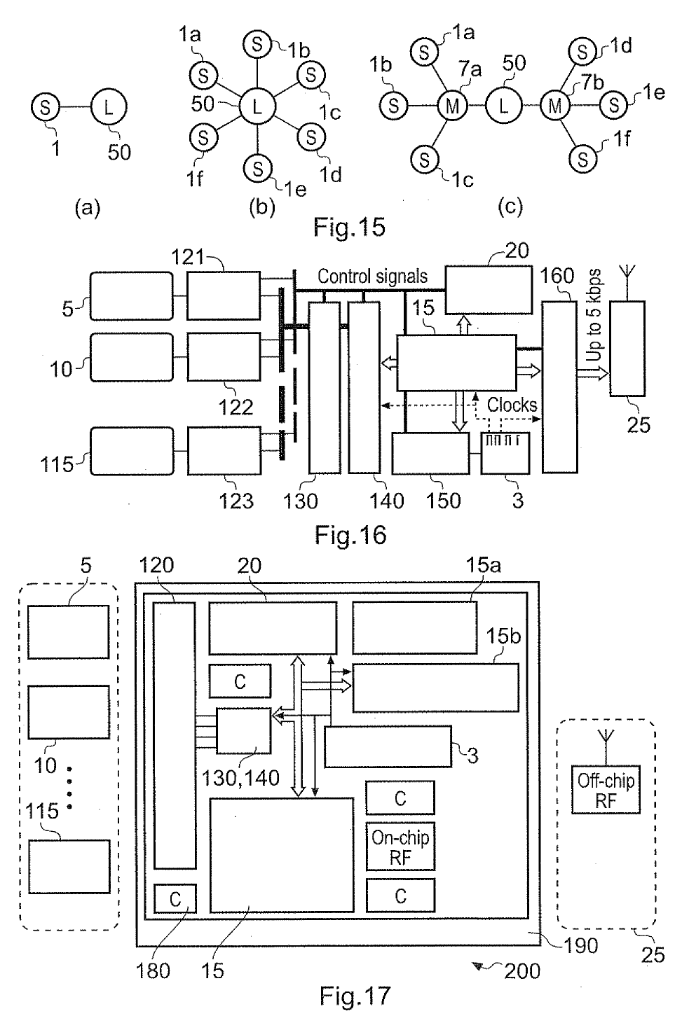

The structure and functionality of the first module will now be described in more detail with reference to FIGS. 16 and 17. FIG. 16 is a block by block diagram of the components and data flow in the first module 1. There are N sensors, of which a first sensor 5, second sensor 10 and Nth sensor 115 are shown. These are linked to a multiplexer 130 via respective sensor circuits 121, 122, 123. The multiplexer 130 multiplexes the signals from the sensor circuits 121, 122, 123 to an ADC 140. The ADC 140 then inputs signals based on the values measured by the sensors 5, 10, 115 to the processor 15. The processor 15 controls the operation of the first module in accordance with a program stored in the memory 20 (internal and external). Memory 20 may be on-chip RAM. The module may also store sensor data based on the parameter values measured by the sensors 5, 10, 115 in the memory 20. The processor 15 passes sensor data based on the measured sensor values to encoder 160. Encoder 160 encodes the data in a format suitable for transmission via transmitter 170 to the second module 50 (or to an intermediate module 7a, 7b). In this embodiment the encoder is a DS-SS encoder block containing a pseudo-random (PN) noise code generator. The PN code length is controlled by the processor 15 to provide an encrypted multiplication process for data transmission. The PN code can be arranged to make it possible for several first modules to share the same base station, using code division multiple access. Operation of the processor 15 and data flow between the processor and connected components is regulated by the first module clock 3. The first module may also contain a DAC 150 to enable the processor 15 to control analogue circuitry, such as the sensors or the clock 3.

FIG. 17 is a block diagram showing how the components of the first module are split onto separate chips. The sensor chips 5, 10, 115 may be arranged separately or as one block. Sensor 5 may be, for example, a pH sensor. The processor 15, memory 20 and clock 3 are all integrated onto one chip 200. The clock 3 may be provided separately, however, this option may take up more space. In this embodiment the sensor circuits 121, 122, 123 are combined as one sensor circuit 120 provided on the same integrated chip as the processor 15 and memory 20. This integrated chip also includes a combined multiplexer and ADC unit 130, 140. Dedicated hardware blocks 15a and 15b provide a SPI (serial peripheral interface) and the DS-SS encoder. However, in the general description these hardware blocks are considered to be part of the processor 15. In FIG. 17, “C” represents a decoupling capacitor, and clock signals are represented by thin arrows.

There is also a transmitter circuit 25, which is provided separately from the aforementioned integrated chip 200. In this embodiment the transmitter circuit includes a surface mount coil inductor, which acts as a magnetic coupler. This eliminates the need for a RF antenna, thus saving space. It would alternatively be possible to use an on-chip RF device, integrated onto the chip 200.

It is important to note that in this embodiment the integrated chip 200 is separated from the analogue sensors 5, 10, 115 and the analogue transmitter circuit 25. It is insulated by pad rings 190 and decoupling capacitors 180.

The processor 15 encodes sensor data for transmission according to the Manchester protocol. However, different protocols could be used and will be apparent to a person skilled in the art. The data transmission is asynchronous in that it does not contain any information relating to the time as which the measured sensor values were taken. Furthermore, the transmission by the first module 1 is continuous in that it does not wait for confirmation of reception of a data packet by the base station 50 before sending the next packet. Accordingly, it is not necessary for the first module to have a receiver. Optionally, a receiver 30 can be provided, as shown in the embodiment of FIG. 14, and in this case a synchronous data exchange protocol can be used, but this option is not preferred as the receiver 30 takes extra power and space in the first module.

The sensor data is encoded such that it is transmitted in 192-bit data packets, followed by a 58-bit “zero-period” in which no data is transmitted. This zero-period makes it easier for the base station 50 to confirm the location of each data packet. Each data packet contains two identical 64-bit codes representing sensor data and 64-bit authentication and parity redundancies. Clearly, the exact content and length of the data packet and exact length of the zero-periods can be varied, the above numbers are just given by way of example.

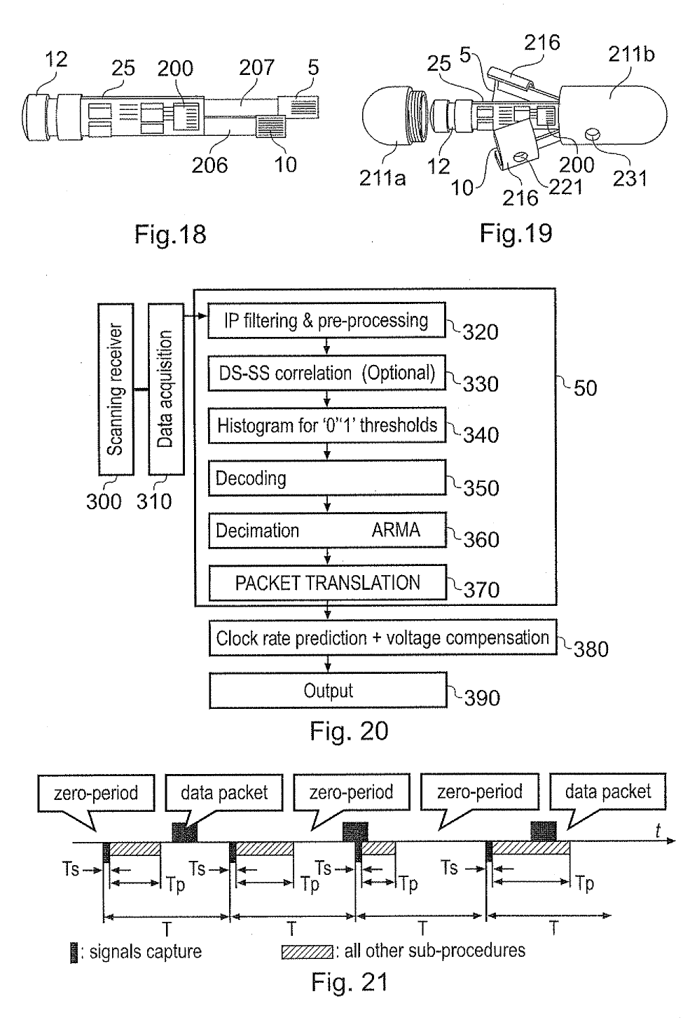

FIG. 18 is a perspective diagram of one embodiment of the first module 1 without its outer casing. Power supply batteries 12 are connected to transmitter 25 and integrated circuit 200 in a line. Flexible cables 206, 207 (e.g. ribbon cables) connect the sensors 5, 10 to the integrated circuit 200. FIG. 19 is a perspective view of the first module with the exterior casing 211 dissembled. It can be seen that, in the FIG. 19 embodiment, the external casing has a first portion 211a which screws into a second casing portion 211b to form the exterior casing 211. The sensors 5, 10 are provided with holder clamps 216 and the flexible cables (e.g. ribbon cables) 206, 207 bend to allow the sensors 5, 10 to be placed in the desired position. The holder clamps 216 have apertures 221 which can be made to align with an aperture 231 in the exterior casing, so as to provide contact between the sensor and the external environment. When the capsule is assembled, the internal electronic components of the first module are protected from the external environment by the outer casing 211. The module is then in the form of a swallowable capsule and has a size approximately equal to a large vitamin pill.

The second module 50 receives the signal transmitted from the first module, which may be in the form of e.g. an on-off-keyed RF signal. It then recovers the data values from the sensors and, because the first module's timing is inaccurate and variable, time-stamps all the sensor values or sets of sensor values using its own clock 23 (which is more accurate and stable than the first module's clock 3). The second module also adjusts the sensor values to compensate for variations in the first module's power supply voltage, as discussed above.

FIG. 20 is a flow chart showing the detailed operation of the second module according to one embodiment. In step 300 the second module's scanning receiver outputs an analogue voltage based on the received transmission frequency within a preset channel bandwidth. This signal contains the transmitted data corrupted by electromagnetic interference.

The second module has a DAQ (Data Acquisition) device, which digitizes this analogue output by over-sampling in step 310. The sample rate is at least twice the Nyquist rate, preferably at least three times the Nyquist rate. The sampling is carried out according to a continuous trigger model, so as not lose any data samples between two sequential signal captures.

As explained above, each “signal” from the first module includes at least a data packet and a “zero period”. Also as explained above, the first module transmits the signals in a continuous stream. For example, a Manchester coded bit-stream with 4 Kbps data rate could be transmitted by the first module and sampled at a 20 KSps over-sampling rate by the second module's DAQ device. FIG. 21 is a time line showing the data packets, zero periods and the time taken for signal capture and the other sub procedures of the FIG. 20 flow chart. The DAQ interval (T as indicated in FIG. 21) is set to be longer than a complete data packet, but shorter than the interval between each data packet. By way of example, one data packet could occupy a 5 KB (e.g. 0.25 s sample interval×20 KSps over-sampling rate×8-bit resolution) or up to 20 KB (e.g. 1 s sample interval×20 KSps×8-bit resolution) local buffer space for an instantaneous process. Of course other sample intervals and rates could be used. In any case, the signal capture (DAQ) procedure should take a relatively short time interval (Ts as indicated in FIG. 21, typically a couple of milliseconds) to complete, so as to leave enough time for the next signal's decoding, packet decimation and packet translation procedures (in time period Tp as indicated in FIG. 10).

After the DAQ step 310, low pass filtering and other pre-processing is carried out on the acquired data samples in step 320. DS-SS correlation is then carried out in step 330 in order to extract the signal sent by the first module 1 from the sampled data. Various possible DS-SS methods will be apparent to a person skilled in the art.

After step 330 the received signal has been converted into a series of digitized analogue values. In step 340 a probability histogram is generated and used to determine a threshold for distinguishing between 0's and 1's. As the threshold can be set adaptively based on the received signal, discrimination between binary values is improved and may be carried out even on a weak signal.

Next, in decoding step 350, the data packets are located and identified and the binary data extracted. It is necessary to do this before processing the data (e.g. sensor values) in each packet. The long ‘zero-period's, during which the communication link is idle, are used to coarsely locate a potential data packet. If the potential data packet actually exists, the pre-defined start sequence (a sequence of one or more start bits) and finish sequence (a sequence of one or more stop bits) are used for precise location of the data packet.

In order to find the data packet, an iteration routine searching from both ends of the signal is employed.

An example of a simple decoding routine for locating the data packets is:

{

0: PointerF=PointerF+stepF; PointerB=PointerB−stepB;

1: Is the Dataset between PointerF and PointerB a valid data packet?

2: If no: go back to 0;

3: If yes: Data packet=Data packet ·x decoding signal;

4: Update the step F and step B;

} * B stands for begin sequence F stands for finish sequence

In addition to the start and stop sequences, characteristics such as bit integrity and bit length can be used to validate the data packet.

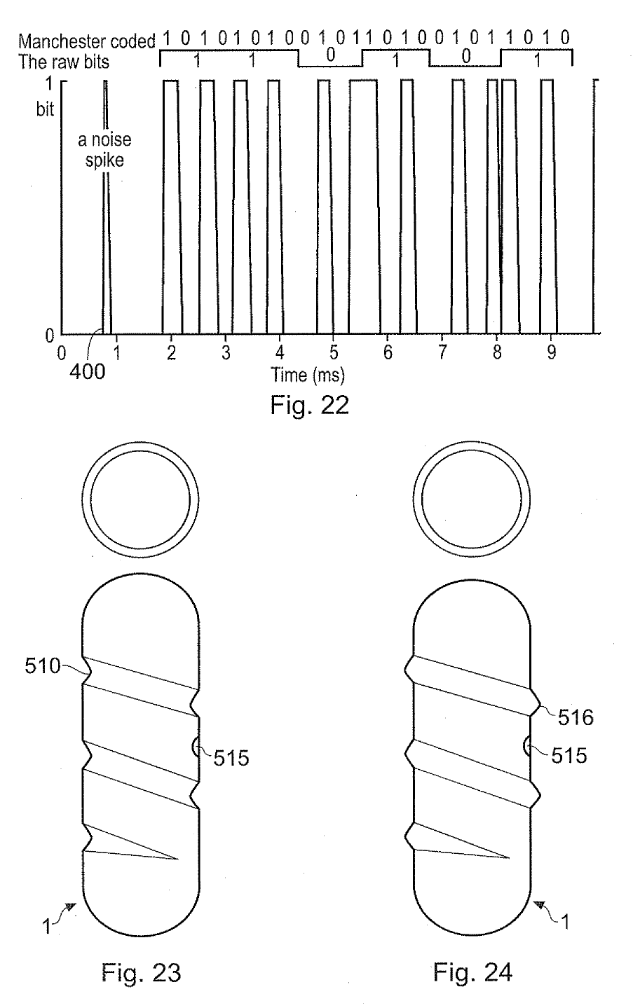

Next in step 360 a median filter, such as an auto-regression moving-average (ARMA) estimator is used to improve the signal to noise ratio. FIG. 22 shows data bits from a portion of a data packet against time, together with a noise spike 400 which is filtered out by the median filtering.

After the median filtering, the data packet is decimated, to get rid of additional data points generated by the over-sampling. The output at this stage, after decimation, includes the data information bits which constitute the complete data packet. Many different formats could be used for the data, one possible example is given below:

Segment 1: Begin sequence (transmitted left to right) 0, 1, 0, 1, 0, 1, 0, 1

Segments 2 to 7: 48 bits of data

Segment 8: Finish sequence 1, 0, 1, 0, 1, 0, 1, 0

----------------------------------------------------------

sub-packet I

Segment 9: Begin sequence 0, 1, 0, 1, 0, 1, 0, 1

Segments 10 to 15: 48 bits of data

Segment 16: Finish sequence 1, 0, 1, 0, 1, 0, 1, 0

----------------------------------------------------------

sub-packet II

Segment 17: Begin sequence 0, 1, 0, 1, 0, 1, 0, 1

Segments 18 to 23: 48 bits of data

Segment 24: Finish sequence 1, 0, 1, 0, 1, 0, 1, 0

----------------------------------------------------------

sub-packet III

In the above example, sub-packets I and II contain sensor data and sub-packet III contains parity data.

Next, in step 370, a packet translation routine extracts the sensor data from the data packet and stamps it with time information based on the time at which the data packet was received by the second module 50, according to the second module's clock 23. The packet translation routine 370 also checks the parity data (e.g. sub packet III) to make sure that the sensor data has been recovered accurately. If the parity and any other authenticity checks are positive an indicator bit is set to ‘1’ to indicate that the data is valid, otherwise the indicator bit is set to ‘0’. The output from this step is the timestamp, the sensor data and the indicator bit. The time stamp may be for each portion of sensor data (of predetermined length, e.g. each sensor value) in the data packet or for the data packet as a whole.

Next, in step 380, the clock rate of the first module's clock 3, at the time that the data packet was transmitted, is estimated. In this embodiment, the clock rate is estimated on the basis of the known number of first module clock cycles, which it takes the first module to produce and transmit a data packet, and the times—according to the second module's clock—at which the start and end of the data packet arrived at the second module. Other embodiments may use different methods of estimating the first module clock rate.

The voltage supplied by the first module's power supply 12 at the time at which sensor data was gathered by sensors 5,10 is then estimated based on a predetermined relationship between the voltage (V) supplied by the first module's power supply 12 and the clock rate (f) of the first module's clock 3. This predetermined relationship may have been determined empirically or theoretically. In one experiment for one first module, the relationship was found to be:

V=A log₁₀f+B

where A=2.35 and B is a constant which is not needed in the compensation procedure.

Once the power supply voltage (V) has been determined, the sensor data values in the sensor data are adjusted to compensate for variations in the power supply voltage. This compensation is carried out on the basis of a predetermined relationship between the sensor values (i.e. the sensor data values which are transmitted by the first module) and the power supply voltage. Generally the sensor values transmitted by the first module, will be based on analogue output from the sensors 10, 15 and the response of the ADC 140 (and any amplifiers) to this output, plus any adjustment made by the first module's processor 15. In many cases the relationship between the sensor values and the power supply voltage will be a linear one. The relationship may be determined theoretically or empirically. Once it is known, it may be used together with the estimated power supply voltage to compensate the sensor data values for variations caused by variations in the power supply voltage.

The second module's processor 70 may also compensate the sensor data values from the first sensor 5 on the basis of the sensor data values taken during the same or a corresponding time period by the second sensor 10. For example if the first sensor 5 is a pH sensor and the second sensor 10 is a temperature sensor, then the sensor data values from the first sensor 5 can be compensated in accordance with the known variation in response of the pH sensor 10 at different temperatures.

Finally, in step 390 the processed sensor data is output to a display, to memory or to a remote device. The output includes the compensated sensor values and the estimated time at which these values were measured.

The second module's processor 70 may also be configured to predict the location of the next data packet, on the basis of the estimated clock rate of the first module's clock 3 and/or the previous estimated clock rates and/or the (time) position of previous data packets. This prediction of packet location can be used to optimize the decoding routine, which searches for data packets, and to help prevent loss of contact between the first and second modules.

The swallowable capsule 1 shown in FIGS. 18 and 19 has an exterior casing with a smooth outer surface. It is however, possible to have an exterior casing with a helical pattern on its outer surface. This helical pattern causes the sensing device to rotate as it passes through the intestinal tract, in a similar manner to a bullet propelled down a rifled gun barrel. In the case of a capsule traveling through the gut, the forward propulsion may be provided by the peristaltic motion of the gut. The helical pattern should be at least one helical turn and may be formed by an indentation, protrusion or groove in or on the capsule's exterior casing. FIG. 23 is a view, from above of a swallowable capsule 1 with two and half helical turns formed by a groove 510 in the exterior casing. FIG. 24 is a view, from above, of a swallowable capsule 1 with two and half helical turns formed by a protrusion 516 on the surface of the exterior casing. Both capsules have an aperture 515 for allowing fluid in the surrounding environment to come into contact with a sensor in the capsule.

The above descriptions with respect to FIGS. 1-24 relate to sensor devices and systems at the system level of operation, in particular with respect to the communication between the first module and the second module and the calibration of the sensors and the interpretation of the sensor output (either by the processor of the first module or by the second module).

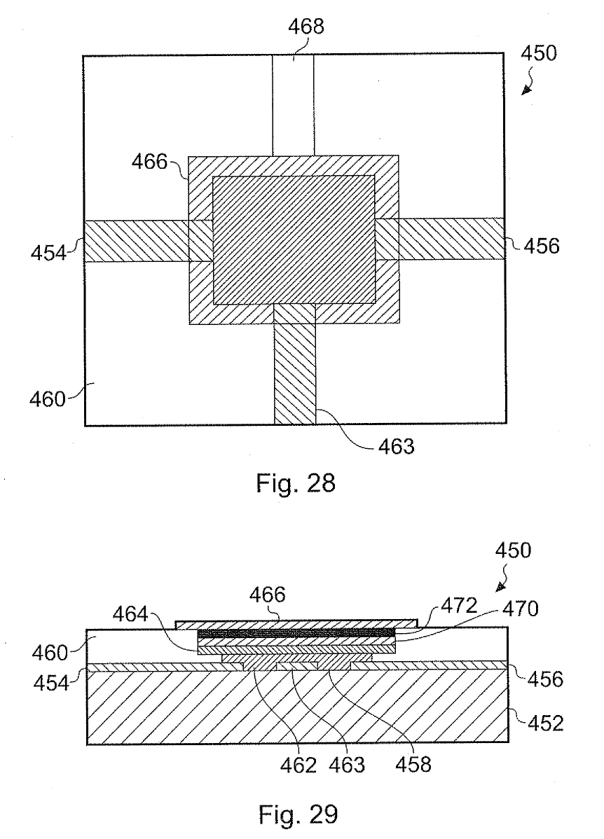

With reference to FIGS. 28 and 29, there is shown in these drawings a sensor element 450 for use in a particular embodiment. The sensor element 450 is a “one-shot” sensor element, in that it can be operated to sense the presence or absence of an analyte only once. As shown in the cross-section view of FIG. 29, the sensor element is formed on a substrate 452. Electrodes 454, 456 and 463 (working electrode 454, counter electrode 456 and reference electrode 463) are formed on top of the substrate 452 but do not meet, being separated by a gap 458. These electrodes are formed of gold, or gold-platinum alloy, or platinum. Typically, the electrodes are formed of different materials, the working electrode being formed of a material selected to catalyze an oxygen redox reaction at its surface (e.g. platinum or gold). The working electrode is typically formed of silver. The purpose of the reference electrode (as will be well understood by the skilled person) is to provide a stable voltage at the working electrode, in order to compensate for the effects of the redox reaction on the working electrode. An insulating layer 460 is formed over the substrate and electrodes, leaving a portion of each of the working electrode 454, counter electrode 456 and reference electrode 463 exposed in a well. The well has a stepped shape, due to a step in the wall of the insulating layer 460. At the base of the well, covering and in contact with the exposed parts of the working, counter and reference electrodes, and filling the gap 458 between the electrodes, is an electrolyte 462. In an embodiment, the electrolyte is an ionically conducting gel or solid electrolyte, such as a solid polymer electrolyte (e.g. polyethylene oxide, a fluorinated sulfonic acid copolymer such as Nafion™ of DuPont). Covering the electrolyte is a semi-permeable membrane 464 that is impermeable to water and electrolyte but permeable to oxygen. Typically the semi-permeable membrane is formed from Teflon™. Extending across the well formed in the insulating layer 460 is a protective layer 466. Typically, this is a gold or gold alloy layer, of thickness about 0.2-0.3 μm. An electrode 468 connects to the protective layer 466.

The space between the protective layer 466 and the semipermeable membrane 464 is a reagent space. In this reagent space a first reagent layer 470 and a second reagent layer 472 are provided. It is possible to arrange the first and second reagents in different configurations, such as in multi-layer form, or as islands of one reagent in the other, or as intimately mixed reagents. The optimum arrangement will depend on the reactivity of the reagents with each other in the absence and presence of the catalytic component, which will be described later.

In the present embodiment, the sensor element 450 is a blood sensor. Haemoglobin (a component of blood) catalyses oxidation of a phenolic compound in the first reagent by a mediator or oxygen donor present in the second reagent. The first reagent in this embodiment is, or contains, alpha guaiaconic acid. An alternative for the first reagent is tetramethylbenzidine (TMB). The second reagent is, or contains iodate or periodate. An alternative for the second reagent is 2,5-dimethylhexane-2,5-dihydroperoxide as an oxygen donor. A further alternative is hydrogen peroxide.

The different layers can be applied to the substrate 452 by known fabrication techniques. For example, spin casting can be used, especially if the substrate 452 is flat, e.g. a silicon substrate. Suitable spin-casting can be achieved in combination with a photo-mask or via a mask and etch process. Etching can be carried out using an oxygen plasma, since guaiac is organic. However, other deposition techniques can be used, such as sputtering, thick film deposition, injection moulding, evaporation, deposition using micro-pipette, etc. As an example, the deposition of guaiac resin into the reagent space can be performed by dissolving the resin in alcohol (e.g. ethanol, N-methyl-2-pyrrolidone (NMP) or dimethylsulphoxide (DMSO)) and then spin casting the solution.

The insulating layer 460 may be formed of polyimide or SU-8.

In use, the sensing element is inactive and remains protected by the protective layer 466 until activation. In order to activate the sensing element, a voltage is applied to the protective layer 466 via electrode 468. A suitable voltage is +1.0V (or higher). A cathode (not shown) is provided elsewhere to complete an electrochemical circuit. The cathode can be formed of any conducting or electroactive material that does not produce toxic electrolysis products. When the sensing element is in an environment of aqueous chloride ions (e.g. the GI tract), the application of this voltage to the protective layer causes corrosion of the protective layer by the formation of chlorogold complexes. These are reduced at the cathode. The protective layer can be removed in as little as 10-30 seconds by this mechanism, with the resultant exposure of the first and second reagents to the environment. The removal of a gold protective layer in this way has been demonstrated by Santini et al, in “Microchips as controlled drug-delivery devices”, Angew. Chem. Int. Ed. 2000, 39, 2396-2407, the content of which is incorporated herein by reference.

Once exposed to the environment of the GI tract the presence of blood in the GI tract causes the catalysis of the reaction between the first and second reagents. The reaction between the first and second reagent produces, as a final end product, dissolved oxygen, optionally by reactive intermediates, depending on the particular reaction taking place and on the solution conditions. The semi-permeable membrane is permeable to oxygen. The electrochemical cell formed in the electrolyte space of the sensor element is, in effect, a Clark cell, as will be well understood by the skilled person. The cell controls or monitors a redox reaction between the working electrode and the counter electrode. In this way, the reaction between the first and second reagents can be monitored and thus a measure of the concentration of the analyte (blood) reaching the sensor element can be obtained.

In another embodiment, the Clark cell is replaced by an optoelectronic detector, in which light from an LED (preferably a white LED) is passed through the reagent space. The optoelectronic detector is capable of detecting a colour change in the reagent space when the first and second reagent react in the presence of blood to produce a blue-green colour. Alternative colour changes could of course be monitored in a similar way, e.g. where different reagents are used.

In certain circumstances (e.g. dependent on temperature and/or pH), the reaction rate between the first and second reagents will vary, even in the absence of blood. Thus, depending on the storage history of the sensor element, and the usage history of the sensor element (e.g. how long it has been in the body), the output from the sensor element (i.e. the potential between the working electrode and counter electrode) will vary, either before activation or after activation. Thus, the sensor element may be calibrated according to one of the schemes and routines set out above, e.g. based on the output of a pH sensor and/or a temperature sensor and/or a measure of the time for which the sensor element has been deployed.

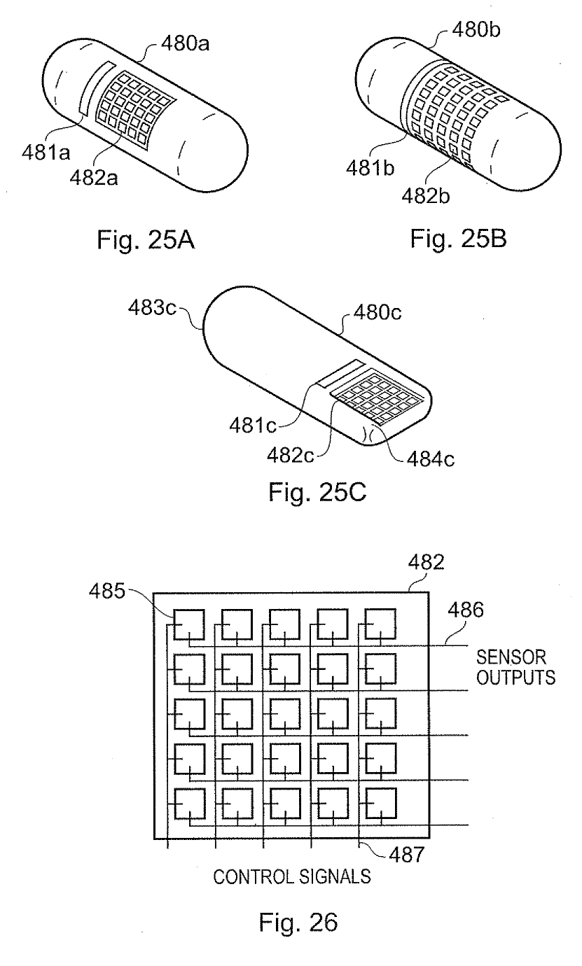

The sensor element can only be activated once, to take a single measurement. Thus, a sensor device is provided which has an array of similar sensor elements. A schematic view of a suitable sensor device 480a and sensor element array 482a is shown in FIG. 25A. In this example, the sensor element array 482a has a curved form and is situated at a curved external surface of the sensor device. Common cathode 481a is also situated at the external surface of the device, for completing the electrochemical circuit required to remove protective film 466 on activation of each sensor element. The sensor array is preferably manufactured in flat form on a flexible substrate (e.g. polyimide) and then flexed to fit the curved outer profile of the sensor device. However, it would also be possible for the sensor element array to be formed in a flat configuration and placed at a flat (or less curved) part of the sensor device. An example of this is shown in the alternative sensor device of FIG. 25C, in which the sensor device 480c has an asymmetric shape, being rounded at one end 483c and flattened at the other end 484c, the sensor array 482c being located at the flattened end 484c. The common cathode 481c can be located at a convenient location, as desired. In alternative embodiments, for example, the sensor device could have a flat form at the longitudinal middle part of the device, or could have a flattened end, or a faceted end in which a flat surface is formed at an inclined angle to the principal axis of the device. Alternatively, the sensor element array can extend substantially fully around the circumference of the sensor device. This is preferred, since this will allow the sensor elements to sample more of the environment of the device. This is illustrated in FIG. 25B, in which the sensor device 480b has a rounded cylindrical shape and the sensor array 482b extends circumferentially around the outer surface of the device, along with the common cathode 481b. As already mentioned, it is possible to provide the sensor element array on a flexible polyimide substrate in flat form, and then flex the substrate to fit it to the device. Alternatively, it is possible to provide the sensor element array as part of the outer casing of the sensing device. For example, suitable well shapes can be molded-in or micro-machined into the outer casing of the device, and/or electrodes can be cast into the outer casing.

FIG. 26 shows a schematic view of a 5×5 sensor element array. Each sensor element 485 has two types of electrical connection—control signal inputs 487 and sensor outputs 486. These connections are only shown schematically in FIG. 26. The control signal inputs for each sensor element consist of an electrical connection to protective layer 466. The sensor outputs 486 actually consist of three electrical connections per cell—one each for the working electrode, counter electrode and reference electrode. The skilled person will readily understand that signal from and to these electrodes can be controlled by similar control and op amp circuitry as already described with reference to the earlier drawings.

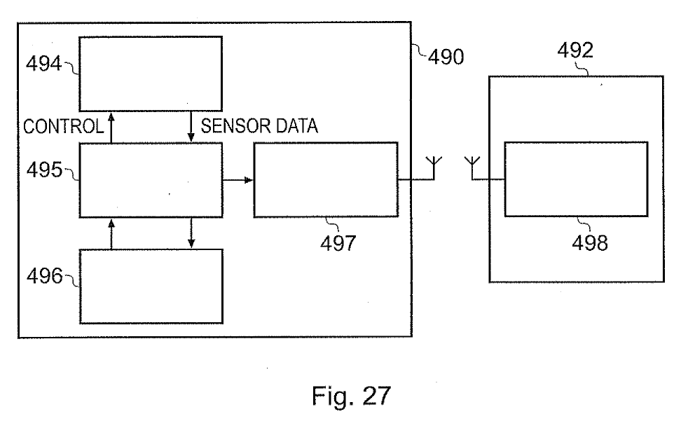

FIG. 27 is a schematic diagram showing a sensing system of a first module 490 and a second module 492. The arrangement is similar in functional terms to that of FIG. 2 except that the first sensor 494 is a sensor element array, such as an array of bio-sensors as has already been described. The controller 495 controls (i.e. activates) one (or more) of the sensor elements at a time, in response to either the output of the second sensor 496 (e.g. a pH sensor or temperature sensor as already described) or as a result of a predetermined timing schedule stored in or available to the controller 495. The sensor output (voltage between the working electrode and counter electrode of the activated sensor element) is detected by the controller 495. Sensor data derived from this output is then transmitted by transmitter 497 of the first module to receiver 498 of the second module.

The operation of all of the sensor elements in the sensor array in sequence is relatively power-consuming, in particular the activation of each sensor element by removal of the protective layer 466 and the application of suitable potential difference between the working electrode and counter electrode. In order to reserve enough power to operate each of the sensor elements adequately throughout the service life of the sensor device (e.g. 19-24 hours), the various power-saving and space-saving measures described with respect to the earlier embodiments are also applied to the present embodiment.

The above-disclosed subject matter is to be considered illustrative, and not restrictive, and the appended claims are intended to cover all such modifications, enhancements, and other embodiments which fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

1. A sensing apparatus including a first module and a second module, said first module having a controller, a transmitter and an array of sensor elements, said controller being capable of activating one or more sensor elements in said array independently of others in the array, in order to obtain a sensor output from said array at different times by using different sensor elements in said array, said transmitter being configured to transmit sensor data, derived from said sensor output, from said first module to a receiver of said second module, wherein each sensor element is a biological sensor for detecting the presence of the same analyte in the environment in which the sensor array is to be deployed.

2. A sensing apparatus according to claim 1 wherein the first module is adapted:
(i) to be swallowable, for passage through the human or animal body;
(ii) to be implantable in the human or animal body; or
(iii) to be placed at a surface location of the human or animal body (e.g. wound site)

3. A sensing apparatus according to claim 1 or claim 2 wherein each sensor element is activatable only once to attempt to detect the presence of said analyte in said environment.

4. A sensing apparatus according to any one of claims 1 to 3 wherein said sensor output corresponds to an analyte condition of at least one of:
analyte present; analyte not present; a quantitative measure of the concentration of analyte detected.

5. A sensing apparatus according to any one of claims 1 to 4 wherein said analyte is blood, or haemoglobin, or another component of blood or a degradation product of blood.

6. A sensing apparatus according to any one of claims 1 to 5 wherein activation of a sensor element in said array allows analyte present in the environment of the sensor element to catalyse a chemical reaction between a first reagent and a second reagent, detection of said chemical reaction by said sensor element determining the sensor element output.

7. A sensing apparatus according to claim 6 wherein each sensor element includes a reagent space containing at least said first reagent.

8. A sensing apparatus according to claim 7 wherein said reagent space also contains said second reagent.

9. A sensing apparatus according to claim 8 wherein said second reagent is in contact with said first reagent.

10. A sensing apparatus according to any one of claims 7 to 9, said reagent space being separated from an electrolyte space by a semi-permeable membrane, said electrolyte space having a working electrode, a counter electrode and optionally a reference electrode, said electrodes being in electrical contact with electrolyte in said electrolyte space.

11. A sensing apparatus according to any one of claims 7 to 10, said reagent space being exposable to said environment on activation of said sensor element.

12. A sensing apparatus according to claim 11, each sensor element including a cover member for covering said reagent space, said cover member being at least partially removable to allow exposure of said reagent space.

13. A sensing apparatus according to claim 12 wherein said cover member is at least partially removable by application of an electrical voltage to said cover member.

14. A sensing apparatus according to claim 13 wherein said electrical voltage triggers at least one of corrosion, dissolution, melting, sublimation and breakage of said cover member.

15. A sensing apparatus according to any one of claims 6 to 14 wherein said first reagent comprises alpha guaiaconic acid or derivative thereof.

16. A sensing apparatus according to any one of claims 6 to 15 wherein the second reagent is a mediator capable of oxidising the first reagent in the presence of a catalyst.

17. A sensing apparatus according to any one of claims 1 to 16 wherein said sensor array is provided at an outer surface of said first module, so as to be provided in contact with the environment in which the first module is to be deployed.

18. A sensing apparatus according to any one of claims 1 to 17 wherein said array includes at least four sensor elements.

19. A sensing apparatus according to any one of claims 1 to 18, wherein said array includes at least nine sensor elements.

20. A sensing apparatus according to any one of claims 1 to 19 wherein said controller is operable to activate said sensor elements at predetermined time intervals.

21. A sensing apparatus according to any one of claims 1 to 20 wherein the sensor array of the first module forms a first sensor and the first module further includes a second sensor, said second sensor being operable to measure a parameter of the environment in which the first module is to be deployed.

22. A sensing apparatus according to claim 21 wherein the output of the second sensor is used by the controller to determine the time at which a sensor element of the sensor array is activated.

23. A sensing apparatus according to claim 21 or claim 22 wherein the first module further includes a third sensor, said third sensor being operable to measure a parameter of the environment in which the first module is to be deployed, different to the parameter measured by the second sensor.

24. A sensing apparatus according to claim 23 wherein the output of both the second and third sensors is used by the controller to determine the time at which a sensor element of the sensor array is activated.

25. A sensing apparatus according to claim 23 or 24 wherein the second and third sensors are selected from:
a pH sensor, a temperature sensor, a dissolved oxygen sensor, a conductivity sensor, a biochemical sensor, an optical sensor and an acoustic sensor.

26. A method of operating a sensing apparatus including a first module and a second module, said first module having a controller, a transmitter and an array of sensor elements, the method including the steps of:
(i) said controller activating at least one sensor element in said array independently of others in the array, so as to obtain a sensor output from said at least one sensor element at a first time t1;
(ii) said controller activating at least one further sensor element in said array independently of others in the array, so as to obtain a sensor output from said at least one further sensor element at time t2, different to t1; and
(iii) transmitting sensor data from said first module to a receiver of said second module,
wherein each sensor element is a biological sensor for detecting the presence of the same analyte in the environment in which the sensor array is to be deployed.

27. A method according to claim 26, further including the step of the controller activating said sensor elements sequentially at different times t so as to obtain a sequence of sensor outputs from said array, corresponding to the detection or absence of said analyte in said environment at said different times t.

28. A method according to claim 26 or claim 27 wherein each sensor element is activated a maximum of one time only, to attempt to detect the presence of said analyte.

29. A sensing device designed for passage through the digestive system of a human or animal body, or implantation into a human or animal body, the device having a first sensor for measuring a first parameter, electronic circuitry or software for calibrating the first sensor in accordance with a calibration routine and a transmitter for transmitting data derived from the first sensor's output to an external device, wherein said circuitry is configured to calibrate the sensor by varying the gain of a variable gain amplifier connected to the sensor and/or by varying an offset voltage applied to the sensor or by varying an offset voltage applied to an amplifier connected to the sensor.

30. A sensing device according to claim 29 wherein the device is a swallowable capsule.

31. A sensing device according to claim 29 or claim 30 wherein the calibration routine is a routine for optimising the dynamic range of the sensor.

32. A sensing device according to any one of claims 29 to 31 wherein the calibration routine comprises the step of determining a relationship between the sensor output and the actual physical value of the measured parameter.

33. A sensing device according to any one of claims 29 to 32 wherein the calibration routine is a routine in which the sensor or surrounding circuitry is adjusted until the sensor has zero output.

34. A sensing device according to any one of claims 29 to 32 wherein said calibration routine is a routine for compensating for drift of said first sensor output over time, the compensation being carried out in accordance with a model of sensor drift over time.

35. A sensing device according to claim 34 wherein the model of sensor drift over time is a predetermined model stored in a memory.

36. A sensing device according to claim 34 wherein the model of sensor drift is calculated while the sensor is in use, by extrapolating previous data points measured by the sensor.

37. A sensing device according to any one of claims 34 to 36 wherein the sensor output is adjusted at regular intervals according to said model in order to compensate for sensor drift.

38. A sensing device according to any one of claims 29 to 32 wherein the calibration routine is a routine in which the sensor output is adjusted such that it indicates the value of the sensed parameter relative to specified reference value.

39. A sensing device according to any one of claims 29 to 32 wherein the calibration routine is a routine in which the sensor is exposed to a known stimulus and the sensor output is adjusted until it is equal to a predetermined value or within a predetermined range specified for said known stimulus.

40. A sensing device according to claim 39 wherein the sensing device is provided in casing containing a liquid or gel having a known value for the physical parameter which the first sensor is designed to measure, and wherein said calibration routine is configured to calibrate the sensor with reference to the sensor's output in response to measuring said liquid or gel.

41. A sensing device according to any one of claims 29 to 40 wherein the sensing device is configured to transmit calibration data to an external device.

42. A sensing device according to any one of claims 29 to 41 wherein the sensing device is configured to carry out said calibration autonomously without reference to instructions or data from an external electronic device.

43. A sensing device according to any one of claims 29 to 41 wherein said sensing device has a receiver for receiving control instructions and/or calibration data from an external device and is configured to carry out said calibration with reference to said control instructions and/or calibration data received from an external device.

44. A sensing device according to any one of claims 29 to 43 wherein the first sensor is a pH sensor, a temperature sensor, a blood sensor, a dissolved oxygen sensor, a conductivity sensor, a biochemical sensor, an optical sensor or an acoustic sensor.

45. A sensing device according to any one of claims 29 to 44 wherein the first sensor comprises an ISFET.

46. A sensing device according to any one of claims 29 to 45 wherein the sensing device's transmitter is a radio transmitter, an induced magnetic field transmitter or an acoustic transmitter.

47. A sensing device according to any one of claims 29 to 46, further comprising a second sensor for measuring a second parameter different to said first parameter, and wherein the calibration routine is configured to adjust the output of said first sensor on the basis of a reading from said second sensor.

48. A sensing device according to claim 47, further comprising a controller for switching on the first sensor when the output from the second sensor displays a predetermined characteristic, or switching on the first sensor a set period of time after the output from the second sensor displays said predetermined characteristic.

49. A sensing device according to claim 47 or claim 48 wherein said first sensor is a blood sensor and said second sensor is a pH sensor.

50. A sensing device according to claim 48 wherein the controller is configured to switch on said first sensor autonomously without input from an external device.

51. A sensing device according to any one of claims 29 to 50, further comprising a processor configured to detect a characteristic event in the first sensor output indicating that the sensing device is at a particular location in the body and to store in a memory and/or transmit to an external device, location data indicating the location of the sensing device.

52. A sensing device according to claim 51 wherein the first sensor is a pH sensor.

53. A sensing device according to claim 51 or claim 52 wherein the processor is configured to detect that the sensing device has left the small bowel and entered the large bowel when the output from the first sensor indicates that the pH has switched from an acidic pH to an alkaline pH.

54. A system for measuring a parameter comprising a first module in the form of a sensing device according to any one of claims 29 to 53 and a second module comprising a receiver for receiving data transmitted by said first module's transmitter.

55. A system according to claim 54 wherein said first module further comprises a receiver for receiving instructions and/or data from said second module; said second module further comprises a transmitter for sending instructions and/or data to said second module and a processor and wherein said processor of the second module is configured to send calibration instructions and/or calibration data to said first module and said first module is configured to calibrate the first sensor on the basis of said received instructions and/or data.

56. A system for measuring a parameter comprising a first module in the form of a sensing device for use in a human or animal body having a first sensor for measuring a first parameter and a transmitter for transmitting measurements made by said first sensor and calibration data generated by said first module to a second module; the second module comprising a receiver for receiving data output by said first module's transmitter, and a processor for processing said data, wherein said second module's processor is configured to calibrate the measurements made by the first sensor in accordance with a calibration routine and on the basis of calibration data sent by said first module.

57. A system according to claim 56 wherein said calibration routine is a routine for compensating for drift of said first sensor output over time, the compensation being carried out in accordance with a model of sensor drift over time.

58. A system according to claim 56 wherein the calibration routine is a routine for relating the first sensor output to an actual physical value of the measured parameter.

59. A system according to claim 56 having a sensing device according to claim 48 wherein the controller is configured to carry out said adjustment of the output of the first sensor, on the basis of the second sensor reading, in response to instructions sent by the second module.

60. Apparatus for gathering data comprising:
a first module suitable for placement inside or passage through a human or animal body, the first module comprising a first clock, at least one sensor, a power supply for supplying power to said first clock and said at least one sensor and a transmitter for transmitting sensor data from said at least one sensor; and
a second module comprising a second clock, a receiver and a processor configured to receive data sent from said first module's transmitter, estimate the first clock's clock rate and compensate the received sensor data for variations in the power of said first module's power source by adjusting the sensor data on the basis of said estimated first clock rate.

61. Apparatus according to claim 60 wherein the first module's transmitter is a radio transmitter and the second module's receiver is a radio receiver.

62. Apparatus according to claim 60 or claim 61 wherein the first module is a swallowable capsule or an implant device for insertion into the large bowel having an aperture for allowing passage of body fluids.

63. Apparatus according to any one of claims 60 to 62 wherein the first module's at least one sensor outputs a series of sensor values each corresponding to a sensor reading taken at a respective time, and wherein for each respective sensor value, the second module's processor estimates the first clock's clock rate at the time when said sensor value was taken and adjusts each respective sensor value to compensate for variations in power from said first module's power supply.

64. Apparatus according to any one of claims 60 to 63 wherein the clock rate of the first clock is estimated on the basis of the rate at which data from the first module is received by the second module.

65. Apparatus according to any one of claims 60 to 64 wherein the compensation is carried out on the basis of a predetermined relationship between the sensor and the voltage supplied by the power supply and a predetermined relationship between the clock rate of the first clock and the voltage supplied to the first clock by the power supply.

66. Apparatus according to any one of claims 60 to 65 wherein the sensor data is transmitted by the transmitter according to a protocol in which said data is split into one or more data packets, each data packet having a fixed predetermined length and wherein each data packet is separated from other data packets by a period of no signal transmission, having a fixed predetermined length.

67. Apparatus according to claim 66 wherein each data packet has a start sequence of one or more bits marking the start of the data packet and a stop sequence of one or more bits marking the end of the data packet.

68. Apparatus according to any one of claims 60 to 67 wherein signal transmission from said first module to said second module is asynchronous.

69. Apparatus according to any one of claims 60 to 68 wherein the at least one sensor is selected from a temperature sensor, a camera, a blood sensor, a pH sensor, a dissolved oxygen sensor, a conductivity sensor and a pressure sensor.

70. Apparatus according to any one of claims 60 to 69 wherein the first module does not have a regulator for regulating the voltage output from the first module's power supply.

71. Apparatus according to any one of claims 60 to 70 wherein the first clock is a low Q clock having a value of Q less than 20.

72. Apparatus according to any one of claims 60 to 71 wherein the first module's transmitter transmits according to a CDMA system and wherein there are a plurality of said first modules, each transmitting on a different channel.

73. Apparatus according to any one of claims 60 to 72 wherein the processor is configured to pre-process the analogue signal from the receiver to generate a probability histogram to determine a voltage threshold to distinguish 0s and 1s in the analogue signal.

74. Apparatus according to any one of claims 60 to 73 wherein the first module has a first sensor and a second sensor and the second module's processor is configured to adjust the sensor values in the sensor data from the first sensor based on the sensor values in sensor data from the second sensor.

75. Apparatus according to claim 74 wherein said second sensor is a temperature sensor.

76. Apparatus according to any one of claims 60 to 75 wherein the first module does not have a receiver for receiving data from an external device.

77. Apparatus according to any one of claims 60 to 76 wherein the first module has an exterior casing with one or more grooves for channeling fluids towards one or more openings in the exterior casing.

78. Apparatus according to any one of claims 60 to 77 wherein the first module is a swallowable capsule and comprises an exterior casing having at least one helical groove, protrusion or indentation for causing the capsule to rotate as it passes through the intestinal tract.

79. A method of transmitting and receiving data in a system comprising a first module having a first clock, at least one sensor, a power supply for supplying power to said first clock and said at least one sensor and a transmitter for transmitting sensor data from said at least one sensor and a second module comprising a second clock, a receiver and a processor; the method comprising the steps of transmitting sensor data based on the output of said at least one sensor to the second module's receiver; and using the second module's processor to estimate the first clock's clock rate and compensating the received sensor data for variations in the power of said first module's power supply by adjusting the sensor data on the basis of said estimated first clock rate.
